(19) United States
(12) Patent Application Publication
(10) Pub. No.: US 2022/0000959 A1
(43) Pub. Date: Jan. 6, 2022

(54) APPLE PEEL POLYPHENOL EXTRACT FOR THE PREVENTION AND THE TREATMENT OF NON-ALCOHOLIC FATTY LIVER DISEASE

(71) Applicant: Leahy Orchards inc., Franklin (CA)

(72) Inventors: Emile LEVY, Montreal (CA); Michael LEAHY, Franklin (CA)

(21) Appl. No.: 17/293,557

(22) PCT Filed: Nov. 13, 2019
(86) PCT No.: PCT/CA2019/051614

§ 371 Date: May 13, 2021

Related U.S. Application Data
(60) Provisional application No. 62/760,143, filed on Nov. 13, 2018.

(51) Int. Cl. A61K 36/73; A61P 1/16

(52) U.S. Cl. CPC A61K 36/73; A61P 1/16

(57) ABSTRACT

The present document describes apple peel polyphenolic extract comprising a proanthocyanidin content of at least 15000 μg/g of dry weight, comprising about 30% to about 35% epicatechin content, and pharmaceutical composition comprising the apple peel polyphenolic extract. The present document also describes methods and use of the apple peel polyphenolic extract for preventing or treating conditions such as oxidative stress, inflammation and mitochondrial dysfunction of the liver, insulin resistance, intestinal endothelial tissue injury, non-alcoholic fatty liver disease, non-alcoholic steatohepatitis (NASH), liver fibrosis, liver cirrhosis, and/or inhibition of de novo lipogenesis and β-oxidation of free fatty acids for preventing accumulation thereof in the liver. The present document also describes a process for extraction of at least a polyphenol content from dry apple peel powder.

### CROSS-REFERENCE TO RELATED APPLICATIONS

This application claims priority of U.S. provisional patent application No. 62/760,143, filed on Nov. 13, 2018, the specification of which is hereby incorporated by reference in its entirety.

### BACKGROUND

### (A) Field

The subject matter disclosed generally relates to positive biological effect of a water-based apple peel polyphenol extract obtained using very mild conditions on the prevention and the treatment of Non-Alcoholic Fatty Acid Liver Disease (NAFLD).

### (b) Related Prior Art

### Introduction

Non-alcoholic fatty liver disease (NAFLD) affects a quarter of the adult population and is considered as the world's most common liver disease in Western countries [1-3]. Although obesity constitutes a key player in the development of NAFLD turning into a major component of the metabolic syndrome (MetS) [4], the clinical and economic burden of NAFLD is mainly due to liver-related morbidity and mortality, including non-alcoholic steatohepatitis (NASH), cirrhosis and hepatocellular carcinoma [5,6]. However, it is important not to underestimate the pathogenic risk of developing cardiovascular diseases given the various reports on left ventricular dysfunction and hypertrophy, cardiac valvular calcifications and certain types of cardiac conduction defects [7-9]. On the other hand, growing evidence underlines the capacity of NAFLD to upset extra-hepatic organs and regulatory pathways, which enhances the risk of many chronic diseases, i.e. type 2 diabetes mellitus, and chronic kidney disease [10,11].

The precise mechanisms of NAFLD development and progression remain incompletely understood since most of the pathogenic pathways are both complex and tightly interconnected. Many scientific groups have proposed oxidative stress (OxS) and inflammation as powerful triggers not only for NAFLD etiology, but also for its procession towards fibrosis, NASH and hepatocellular carcinoma [12]. Moreover, the pro-oxidant and inflammatory processes impair insulin signalling, thereby leading to insulin resistance (IR) [13,14], which constitutes the molecular basis for lipid accumulation in the liver and the progression of NAFLD [15].

At present, the management of patients with NAFLD and NASH poses difficult problems and remains a sizable challenge for health providers. The pharmacological approach has fallen far short of expectations, and there is no approved pharmacotherapy [16]. Undoubtedly, large-scale trials are needed to verify treatment effects of new drugs and to provide clinical evidence before their application. Although lifestyle modifications such as vigorous physical activity, balanced diets and weight loss, are regarded as the cornerstone of NAFLD management [1,2,17], this approach faces various complications, including poor adherence and complications. For example, weight loss might aggravate the inflammatory status of subjects with NAFLD [18,19]. Therefore, considerable effort is now directed to explore novel therapeutic agents and especially natural compounds.

Therefore, there is a need for novel approaches for the mitigation or treatment of NAFLD.

### SUMMARY

According to an embodiment, there is provided an apple peel polyphenolic extract comprising
a proanthocyanidin (PAC) content of at least 15000 μg/g of dry weight, comprising
an about 30% to about 35% epicatechin content comprising
   epicatechin momomers and oligomers having from about 1 to 5 epicatechin units, from about 3500 to 4000 μg/g of dry weight;
   epicatechin oligomers having from about 6 to 10 epicatechin units, from about 1000 to 1100 μg/g of dry weight; and epicatechin polymers having >10 epicatechin units, from about 500 to 600 μg/g of dry weight.

The epicatechin oligomers having from about 1 to 5 epicatechin units may comprise epicatechin monomers, from about 700 to 800 μg/g of dry weight.

The epicatechin oligomers having from about 1 to 5 epicatechin units may comprise epicatechin dimers, from about 1000 to about 1200 μg/g of dry weight.

The epicatechin oligomers having from about 1 to 5 epicatechin units may comprise epicatechin trimers, from about 600 to 700 μg/g of dry weight.

The epicatechin oligomers having from about 1 to 5 epicatechin units may comprise epicatechin tetramers, from about 500 to 600 μg/g of dry weight.

The epicatechin oligomers having from about 1 to 5 epicatechin units may comprise epicatechin pentamers, from about 500 to 600 μg/g of dry weight.

The apple peel polyphenolic extract may further comprise a total flavonol content of from about 5000 to about 5400 μg/g of dry weight.

The total flavonol content may comprise a quercetin-glucoside, from about 1600 to about 2000 μg/g of dry weight.

The total flavonol content may comprise a quercetin-arabinoside, from about 950 to about 1150 μg/g of dry weight.

The total flavonol content may comprise a quercetin-xyloside, from about 500 to about 600 μg/g of dry weight.

The total flavonol content may comprise a quercetin-galactoside, from about 300 to about 400 μg/g of dry weight.

The total flavonol content may comprise a quercetin-rhamnoside, from about 1100 to about 1300 μg/g of dry weight.

The total flavonol content may comprise a quercetin, from about 100 to about 200 μg/g of dry weight.

The total flavonol content may comprise a rutin, from about 200 to about 300 μg/g of dry weight.

The total flavonol content may comprise a phlorizin, from about 1200 to about 1400 μg/g of dry weight.

The apple peel polyphenolic extract may further comprise a total phenolic acid content of from about 2000 to about 2400 μg/g of dry weight.

The total phenolic acid content may comprise a chlorogenic acid, from about 2000 to about 2100 μg/g of dry weight.

The total phenolic acid content may comprise a coumaroyl glucoside, from about 10 to about 20 μg/g of dry weight.

The total phenolic acid content may comprise a dihydrobenzoic acid, from about 25 to about 45 μg/g of dry weight.

The total phenolic acid content may comprise a caffeic acid, from about 1 to about 10 μg/g of dry weight.

The total phenolic acid content may comprise a coumaroyl hexose, from about 100 to about 200 μg/g of dry weight.

The apple peel polyphenolic extract may comprise:
a proanthocyanidin (PAC) content of at least 15000 μg/g of dry weight, comprising
an about 30% to about 35% epicatechin content comprising
   epicatechin oligomers having from about 1 to 5 epicatechin units, from about 3500 to 4000 μg/g of dry weight;
   epicatechin oligomers having from about 6 to 10 epicatechin units, from about 1000 to 1100 μg/g of dry weight; and
   epicatechin polymers having >10 epicatechin units, from about 500 to 600 μg/g of dry weight;

a total flavonol content of from about 5000 to about 5400 μg/g of dry weight; and

a total phenolic acid content of from about 2000 to about 2400 μg/g of dry weight.

According to another embodiment, there is provided a pharmaceutical composition comprising the apple peel polyphenolic extract of the present invention, and a pharmaceutically acceptable carrier.

According to another embodiment, there is provided a method of preventing or treating oxidative stress, inflammation and mitochondrial dysfunction of a liver comprising administering to a subject in need thereof a therapeutically effective amount of an apple peel polyphenolic extract according to the present invention, or a pharmaceutical composition according to the present invention.

According to another embodiment, there is provided a method of inhibition of de novo lipogenesis and β-oxidation of free fatty acids for preventing accumulation thereof in a liver comprising administering to a subject in need thereof a therapeutically effective amount of an apple peel polyphenolic extract according to the present invention, or a pharmaceutical composition according to the present invention.

According to another embodiment, there is provided a method of preventing or treating insulin resistance comprising administering to a subject in need thereof a therapeutically effective amount of an apple peel polyphenolic extract according to the present invention, or a pharmaceutical composition according to the present invention.

According to another embodiment, there is provided a method of preventing or treating intestinal endothelial tissue injury comprising administering to a subject in need thereof a therapeutically effective amount of an apple peel polyphenolic extract according to the present invention, or a pharmaceutical composition according to the present invention.

According to another embodiment, there is provided a method of preventing or treating any one of oxidative stress and inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, or combinations thereof, comprising administering to a subject in need thereof a therapeutically effective amount of an apple peel polyphenolic extract according to the present invention, or a pharmaceutical composition according to the present invention.

According to another embodiment, there is provided a method of preventing or treating any one of non-alcoholic fatty liver disease, non-alcoholic steatohepatitis (NASH), liver fibrosis, liver cirrhosis, and combinations thereof comprising administering to a subject in need thereof a therapeutically effective amount of an apple peel polyphenolic extract according to the present invention, or a pharmaceutical composition according to the present invention.

The non-alcoholic fatty liver disease, non-alcoholic steatohepatitis (NASH), liver fibrosis, and liver cirrhosis may comprise steatosis.

The therapeutically effective amount may be from about 100 mg/kg to about 500 mg/kg.

The apple peel polyphenolic extract may be for once daily use, twice daily use, thrice daily use.

The subject may be a human subject.

According to another embodiment, there is provided a use of an apple peel polyphenolic extract according to the present invention, or a pharmaceutical composition according to the present invention, for the prevention or treatment of oxidative stress, inflammation and mitochondrial dysfunction of a liver in a subject in need thereof.

According to another embodiment, there is provided a use of an apple peel polyphenolic extract according to the present invention, or a pharmaceutical composition according to the present invention, for inhibition of de novo lipogenesis and β-oxidation of free fatty acids for preventing accumulation thereof in a liver in a subject in need thereof.

According to another embodiment, there is provided a use of an apple peel polyphenolic extract according to the present invention, or a pharmaceutical composition according to the present invention, for preventing or treating insulin resistance in a subject in need thereof.

According to another embodiment, there is provided a use of an apple peel polyphenolic extract according to the present invention, or a pharmaceutical composition according to the present invention, for preventing or treating intestinal endothelial tissue injury in a subject in need thereof.

According to another embodiment, there is provided a use of an apple peel polyphenolic extract according to the present invention, or a pharmaceutical composition according to the present invention, for preventing or treating any one of oxidative stress and inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, or combinations thereof, in a subject in need thereof.

According to another embodiment, there is provided a use of an apple peel polyphenolic extract according to the present invention, or a pharmaceutical composition according to the present invention, for the prevention or treatment of any one of non-alcoholic fatty liver disease, non-alcoholic steatohepatitis (NASH), liver fibrosis, and liver cirrhosis in a subject in need thereof.

The non-alcoholic fatty liver disease, non-alcoholic steatohepatitis (NASH), liver fibrosis, and liver cirrhosis may comprise steatosis.

The therapeutically effective amount may be from about 100 mg/kg to about 500 mg/kg.

The use may be once daily, twice daily, thrice daily.

The subject may be a human subject.

The apple peel polyphenolic extract according to the present invention, or a pharmaceutical composition according to the present invention, for use in the prevention or treatment of oxidative stress, inflammation and mitochondrial dysfunction of a liver in a subject in need thereof.

According to another embodiment, there is provided an apple peel polyphenolic extract according to the present invention, or a pharmaceutical composition according to the present invention, for inhibition of de novo lipogenesis and β-oxidation of free fatty acids for preventing accumulation thereof in a liver in a subject in need thereof.

According to another embodiment, there is provided an apple peel polyphenolic extract according to the present invention, or a pharmaceutical composition according to the present invention, for preventing or treating insulin resistance in a subject in need thereof.

According to another embodiment, there is provided an apple peel polyphenolic extract according to the present invention, or a pharmaceutical composition according to the present invention, for preventing or treating intestinal endothelial tissue injury in a subject in need thereof.

According to another embodiment, there is provided an apple peel polyphenolic extract according to the present invention, or a pharmaceutical composition according to the present invention, for preventing or treating any one of oxidative stress and inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, or combinations thereof, in a subject in need thereof.

According to another embodiment, there is provided an apple peel polyphenolic extract according to the present invention, or a pharmaceutical composition according to the present invention, for use in the prevention or treatment of non-alcoholic fatty liver disease, non-alcoholic steatohepatitis (NASH), liver fibrosis, and liver cirrhosis in a subject in need thereof.

The non-alcoholic fatty liver disease, non-alcoholic steatohepatitis (NASH), liver fibrosis, and liver cirrhosis may comprise steatosis.

The therapeutically effective amount may be from about 100 mg/kg to about 500 mg/kg.

The use may be once daily, twice daily, thrice daily.

The subject may be a human subject.

According to another embodiment, there is provided a process for extraction of at least a polyphenol content from dry apple peel powder (DAPP) comprising:
1) macerating a first mixture comprising
   from about 10% to about 15% w/w DAPP, and
   water at about 8° C. to about 12° C.,
for a time sufficient and with sufficient mechanical energy to obtain an homogenous mixture and extract a soluble polyphenol content from the DAPP;
2) heating the first mixture to a temperature of about 48° C. to about 52° C., to obtain a second mixture,
3) at least one step of concentration comprising
   i) concentrating the second mixture for concentration of a soluble fraction therefrom,
   ii) filtering the second mixture for removal of a solid fraction therefrom,
   or
   iii) a combination thereof,
to obtain a concentrated polyphenol fraction.

The process may further comprise a step of pasteurizing and/or sterilizing the concentrated polyphenol fraction, to obtain a pasteurized or sterilized concentrated polyphenol fraction.

The water of step 1) may be at a temperature of 10° C.

The at least one step of concentration may be concentrating the second mixture for concentration of a soluble fraction therefrom followed by filtering for removal of a solid fraction therefrom, to obtain a concentrated polyphenol fraction

The heating of step 2) may be up to a temperature of about 50° C.

The DAPP may be about 100 mesh to about 200 mesh, preferably 100 mesh.

The sufficient mechanical energy may be provided by a sheer mixer.

The filtering may be effected with a filter having a pore size of from about 2 μm to about 45 μm, with a filter press, with a solid support filter, a centrifugation, or combination thereof.

The solid support filter may be a diatomaceous earth filter.

The concentrating may be performed with a concentrator unit.

The concentrator unit may be a reverse osmosis unit, a vacuum evaporation unit, or a combination thereof.

The step of concentration may be effected once.

The step of concentration may be effected a plurality of times.

The step of microfiltration may be performed prior to the step of sterilizing.

The process of the present invention may be free of enzyme to digest said DAPP.

The process may be performed in absence of light.

The following terms are defined below.

The term «composition» as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody or fragment thereof, preferably in purified form, together with a suitable amount of carrier so as to provide the form for proper administration to the patient. The formulation should suit the mode of administration.

The terms “preventing”, as used herein in the context of the invention means to slow, hinder, restrain reduce or keep (the condition or disease) from happening or arising.

The term “administering” as used herein refers to any action that results in exposing or contacting a subject with a composition containing an apple peel polyphenolic extract, according to the invention. As used herein, administering may be conducted in vivo, in vitro, or ex vivo. For example, a composition may be administered by injection or through a formulation (i.e. a pharmaceutical composition). Administering also includes the direct application to cells of a composition according to the present invention.

The term “subject” as used herein, is a human patient or other animal such as another mammal.

The term “oxidative stress” as used herein refers to an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA. Oxidative stress from oxidative metabolism causes base damage, as well as strand breaks in DNA. Base damage is mostly indirect and caused by reactive oxygen species (ROS) generated, e.g. O₂⁻ (superoxide radical), OH (hydroxyl radical) and H₂O₂ (hydrogen peroxide). Further, some reactive oxidative species act as cellular messengers in redox signaling. Thus, oxidative stress can cause disruptions in normal mechanisms of cellular signaling.

The term “inflammation” as used herein refers to the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five classical signs of inflammation are heat, pain, redness, swelling, and loss of function. Inflammation is a generic response, and therefore it is considered as a mechanism of innate immunity, as compared to adaptive immunity, which is specific for each pathogen. Too little inflammation can lead to progressive tissue destruction by the harmful stimulus (e.g. bacteria) and compromise the survival of the organism. In contrast, chronic inflammation may lead to a host of diseases, such as hay fever, periodontitis, atherosclerosis, rheumatoid arthritis, and even cancer. Inflammation is therefore normally closely regulated by the body.

The term “mitochondrial dysfunction” as used herein refers to any disruption of the normal function of mitochondria, such as the physiological function of mitochondria in the generation of ATP by oxidative phosphorylation, the generation and detoxification of reactive oxygen species, involvement in some forms of apoptosis, regulation of cytoplasmic and mitochondrial matrix calcium, synthesis and catabolism of metabolites and the transport of the organelles themselves to correct locations within the cell. Abnormality in any of these processes can be termed mitochondrial dysfunction.

The term “de novo lipogenesis” as used herein refers to hepatic de novo lipogenesis which is the biochemical process of synthesizing fatty acids from acetyl-CoA subunits that are produced from a number of different pathways within the cell, most commonly carbohydrate catabolism.

The term “β-oxidation” as used herein refers to the catabolic process by which fatty acid molecules are broken down in the mitochondria in eukaryotes to generate acetyl-CoA, which enters the citric acid cycle, and NADH and FADH₂, which are co-enzymes used in the electron transport chain. It is named as such because the beta carbon of the fatty acid undergoes oxidation to a carbonyl group. β-oxidation is primarily facilitated by the mitochondrial trifunctional protein, an enzyme complex associated with the inner mitochondrial membrane, although very long chain fatty acids are oxidized in peroxisomes.

The term “insulin resistance” is a pathological condition in which cells fail to respond normally to the hormone insulin. Under normal conditions of insulin reactivity, the insulin response triggers glucose being taken into body cells, to be used for energy, and inhibits the body from using fat for energy, thereby causing the concentration of glucose in the blood to decrease as a result, staying within the normal range even when a large amount of carbohydrates is consumed. When the body produces insulin under conditions of insulin resistance, the cells are resistant to the insulin and are unable to use it as effectively, leading to high blood sugar.

The term “intestinal endothelial tissue injury” as used herein refers to damage to the endothelial intestinal tissue that reduces its permeability and causes increase in endotoxin production, for example.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates the changes in anthropometric, metabolic and liver parameters in Psammomys obesus in response to experimental diets and DAPP. Psammomys Obesus animals were exposed to low energy (LE)- and high energy (HE)-diets for 8 weeks. Another group of HE-fed animals was treated with a daily gavage dose (200 mg/kg body weight) of DAPP whereas LE and HE groups were gavaged with vehicle (water). Anthropometric parameters: (A) body weight, (B) liver weight, (C) fatty liver index; Metabolic parameters: (D) glycemia, (E) insulinemia, (F) Homa-IR index, Liver parameters: (G) AST, (H) ALT, (I) liver triglycerides, (J) liver total cholesterol; Insulin sensitivity factors: (K) IRS-1, (L) IRS-2, (M) GSK3β, (N) AMPK were assayed as described in Material and Methods section. Data are expressed as the mean±SEM for 3-5 animals/group. *P<0.05, **P<0.001, ***P<0.0001 vs. low energy (LE) diet Psammomys obesus; P<0.05, ^{##}P<0.001, ^{###}P<0.0001 vs. high energy (HE) diet animal groups.

FIG. 2 illustrates modifications of oxidative stress, antioxidant defense and anti-inflammatory levels in the liver of Psammomys obesus following the administration of experimental diets and DAPP. All the animals on LE, HE and HE+DAPP were sacrificed after 8 weeks of treatment to determine the impact on (A) Peroxide (H₂O₂), (B) malondialdehyde (MDA), (C) Myeloperoxidase (MPO), (D) Oxidized glutathione (GSSG), (E) Reduced glutathione (GSH), (F) GSH/GSSG ratio, (G) Catalase, (H) Glutahione peroxidase (GPx), (I) Tumor necrosis factor alpha (TNF-α), (J) Interleukin beta 1 (IL-1β) and (K) Nuclear factor-erythroid 2-related factor 2 (NRF2) as described in Material and Methods section. Data are expressed as the mean±SEM for 3-5 animals/group. *P<0.05, **P<0.001, ***P<0.0001 vs. low energy (LE) diet Psammomys obesus; ^{#}P<0.05, ^{###}P<0.0001 vs. high energy (HE) diet animal groups.

FIG. 3 illustrates alterations in intestinal permeability, endotoxemia and stool short-chain fatty acid content following experimental diets and DAPP treatment. Next to the consumption of LE, HE and HE+DAPP for consecutive 8 weeks, Psammomys obesus animals were sacrificed to evaluate their effects on (A) fluorescein isothiocyanate (FITC), (B) Zonulin (ZO), (C) Occludin, (D) Plasma trimethylamine N-Oxide (TMAO), (E) Plasma liposaccharide (LPS), (F) Plasma lipopolysaccharide-binding protein (LBP), (G) Liver LPS and (H) short-chain fatty acids SCFA (SCFA) as described in Material and Methods section. Data are expressed as the mean±SEM for 3-5 animals/group. *P<0.05, **P<0.001, ***P<0.0001 vs. low energy (LE) diet Psammomys obesus; ^{#}P<0.05, ^{##}P<0.001 vs. high energy (HE) diet animal groups.

FIG. 4 illustrates the influence of the experimental diets and DAPP treatment on the gluconeogenesis, lipogenesis and fatty acid oxidation pathways in the liver of Psammomys obesus. At the end of 8-week experimental diets, animals were sacrificed and liver was removed to assess the gene expression of (A) Glucose-6-phosphatase (G6Pase) (B) Phosphoenolpyruvate carboxykinase (PEPCK), (C) Peroxisome proliferator-activated receptor beta (PPAR-β), (D) Forkhead box O-1 (FOXO-1), (E) Acetyl-CoA carboxylase (ACC), (F) Fatty acid synthase (FAS), (G) PPAR-γ, (H) Sterol regulatory element binding protein (SREBP) (I) Carnitine palm itoyltransferase 1 alpha (CPT-1α), (J) CPT-1 α activity, (K) Acyl-CoA oxidase 1 (ACOX-1) activity, and (L) PPAR-α mRNA as described in Material and Methods section. Data are expressed as the mean±SEM for 3-5 animals/group. *P<0.05, **P<0.001 vs. low energy (LE) diet Psammomys obesus; #P<0.05, ##P<0.001 vs. high energy (HE) diet animal groups.

FIG. 5 illustrates the effects of the experimental diets and DAPP treatment on mitochondrial functions in the liver of Psammomys obesus. All the animals on LE, HE and HE+DAPP were sacrificed after 8 weeks of treatment to determine in isolated mitochondrial organelles the impact of the diets and DAPP on (A-E) Complexes 1-5 of oxidative phosphorylation system (OXPHOS), (F) ATP, (G) 8-hydroxy-2′-deoxyguanosine (8-OHDG), (H) Peroxisome proliferative activated receptor gamma, co-activator-1 alpha (PGC1α) as described in Material and Methods section. Data are expressed as the mean±SEM for 3-5 animals/group. *P<0.05, **P<0.001, ***P<0.0001 vs. low energy (LE) diet Psammomys obesus; ^{#}P<0.05, ^{##}P<0.001 vs. high energy (HE) diet animal groups.

FIG. 6 illustrates modulation of in vivo and in vitro intestinal fat transport and chylomicron production following experimental diets and DAPP treatment. In vivo lipid transport was assessed following fat meal test in order to estimate increase of (A) Triglyceride (TG) and (B) chylomicron (CM)-TG in the blood circulation. Further, to determine the role of CM in LPS transport, Pluronic L-81 was added to the HE diet, and LPS was measured in (C) Plasma and (D) liver. Intestinal organ culture was performed to delineate the mechanisms of CM assembly, including Apolipoprotein (APO) B-48 synthesis, (F) CM secretion, (G) Microsomal triglyceride transfer protein (MTP), (H) Monoacylglycerol acyltransferase (MGAT) and (I) Diacylglycerol acyltransferase (DGAT) as described in Material and Methods section. Data are expressed as the mean±SEM for 3-5 animals/group. *P<0.05, **P<0.001, ***P<0.0001 vs. low energy (LE) diet Psammomys obesus; ^{#}P<0.05, ^{##}P<0.001 vs. high energy (HE) diet animal groups.

FIG. 7 illustrates a process according to an embodiment of the present invention.

FIG. 8 illustrates a process according to an embodiment of the present invention.

FIG. 9 illustrates, as a chromatogram, an apple peel polyphenolic extract according to an embodiment of the present invention.

It will be noted that throughout the appended drawings, like features are identified by like reference numerals.

### DETAILED DESCRIPTION

An objective of the present work is to determine the effects and mechanisms of action of dried extract of apple peel polyphenols (DAPP) on several processes implicated in NAFLD development and progression. As the gut-liver axis plays a functional role in cardiometabolic disorders (CMD) [22,23], a focus is placed on the delivery of components provided by the intestine to hepatic cells. To get insight into these important aspects, we employed the Psammomys obesus (P. obesus) sand rat, a unique model of MetS [24-36]. To appraise the effects of polyphenolic compounds, we used DAPP, which represents an efficient functional food for the treatment of OxS and inflammation [21,37,38].

Therefore, in embodiments there is disclosed an apple peel polyphenolic extract comprising
a proanthocyanidin (PAC) content of at least 15000 μg/g of dry weight, comprising
an about 30% to about 35% epicatechin content comprising
   epicatechin monomers and oligomers having from about 1 to 5 epicatechin units, from about 3500 to 4000 μg/g of dry weight;
   epicatechin oligomers having from about 6 to 10 epicatechin units, from about 1000 to 1100 μg/g of dry weight; and epicatechin polymers having >10 epicatechin units, from about 500 to 600 μg/g of dry weight.

### Proanthocyanidins

As used herein, the term proanthocyanidins refers to condensed tannins (also known as polyflavonoid tannins, catechol-type tannins, pyrocatecollic type tannins, non-hydrolysable tannins or flavolans). They are polymers formed by the condensation of flavans. They do not contain sugar residues.

They are called proanthocyanidins as they yield anthocyanidins when depolymerized under oxidative conditions. Different types of condensed tannins exist, such as the procyanidins, propelargonidins, prodelphinidins, profisetinidins, proteracacinidins, proguibourtinidins or prorobinetidins. All of the above are formed from flavan-3-ols, but flavan-3,4-diols, called leucoanthocyanidin also form condensed tannin oligomers, e.g. leuco-fisetinidin form profisetinidin, and flavan-4-ols form condensed tannins, e.g. 3′,4′,5,7-flavan-4-ol form proluteolinidin (luteoforolor). One particular type of condensed tannin, found in grape, are procyanidins, which are polymers of 2 to 50 (or more) catechin units joined by carbon-carbon bonds. These are not susceptible to being cleaved by hydrolysis.

While many hydrolysable tannins and most condensed tannins are water-soluble, several tannins are also highly octanol-soluble. Some large condensed tannins are insoluble. Differences in solubilities are likely to affect their biological functions.

The apple peel polyphenolic extract of the present invention may comprise from about 30% to about 35% epicatechin. Catechin is a flavan-3-ol, a type of natural phenol and antioxidant. It is a plant secondary metabolite. It belongs to the group of flavan-3-ols (or simply flavanols), part of the chemical family of flavonoids. Catechin possesses two benzene rings (called the A- and B-rings) and a dihydropyran heterocycle (the C-ring) with a hydroxyl group on carbon 3. The A ring is similar to a resorcinol moiety while the B ring is similar to a catechol moiety. There are two chiral centers on the molecule on carbons 2 and 3. Therefore, it has four diastereoisomers. Two of the isomers are in trans configuration and are called catechin and the other two are in cis configuration and are called epicatechin.

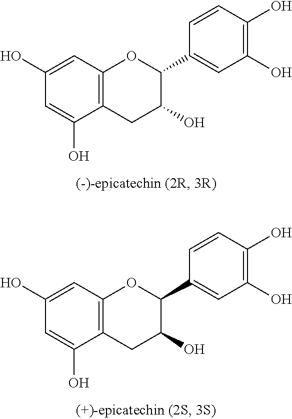 [SMILES: Oc1cc(O)c2c(c1)O[C@H](c1ccc(O)c(O)c1)[C@H](O)C2 ; Oc1cc(O)c2c(c1)O[C@@H](c1ccc(O)c(O)c1)[C@@H](O)C2]

According to embodiments of the present invention, the epicatechin oligomers content of the apple peel polyphenolic extract of the present invention may comprise epicatechin oligomers having from about 1 to 5 epicatechin units, from about 3500 to 4000 μg/g of dry weight; epicatechin oligomers having from about 6 to 10 epicatechin units, from about 1000 to 1100 μg/g of dry weight; and epicatechin polymers having >10 epicatechin units, from about 500 to 600 μg/g of dry weight.

In embodiments, the epicatechin oligomers having from about 1 to 5 epicatechin units may comprise epicatechin monomers, from about 700 to 800 μg/g of dry weight; epicatechin dimers, from about 1000 to about 1200 μg/g of dry weight; epicatechin trimers, from about 600 to 700 μg/g of dry weight; epicatechin tetramers, from about 500 to 600 μg/g of dry weight; and epicatechin pentamers, from about 500 to 600 μg/g of dry weight.

### Flavonols

Flavonols are a class of flavonoids that have the 3-hydroxyflavone backbone. Their diversity stems from the different positions of the phenolic —OH groups. Flavonols are present in a wide variety of fruits and vegetables. In Western populations, estimated daily intake is in the range of 20-50 mg per day for flavonols. Individual intake varies depending on the type of diet consumed.

Besides being a subclass of flavonoids, flavonols are suggested by a study of cranberry juice to play a role along with proanthocyanidins, in the juice's ability to block bacterial adhesion, demonstrated by the compressing of the fimbria of E. coli bacteria in the urinary tract so as to greatly reduce the ability of those bacteria to stay active and initiate an infection. Flavonol aglycones in plants are potent antioxidants that serve to protect the plant from reactive oxygen species (ROS).

The apple peel polyphenolic extract of the present invention may further comprise a total flavonol content of from about 5000 to about 5400 μg/g of dry weight.

The total flavonol content may comprise rutin, quercetin and quercetin glycosides. Quercetin is a plant flavonol from the flavonoid group of polyphenols, is found in many fruits, vegetables, leaves, and grains; red onions and kale are common foods containing appreciable content of quercetin. It has a bitter flavor and is used as an ingredient in dietary supplements, beverages, and foods. The quercetin glycosides include quercetin-glucoside (Isoquercetin-Quercetin-3-O-glucoside), quercetin-arabinoside (Guaijaverin-quercetin-3-O-arabinoside), quercetin-xyloside (quercetin-3-O-xyloside), quercetin-galactoside (Hyperoside-quercetin-3-O-galactoside), and quercetin-rhamnoside (Quercitrin-quercetin-3-O-rhamnoside).

The total flavonol content may comprise quercetin-glucoside, from about 1600 to about 2000 μg/g of dry weight; quercetin-arabinoside, from about 950 to about 1150 μg/g of dry weight; quercetin-xyloside, from about 500 to about 600 μg/g of dry weight; quercetin-galactoside, from about 300 to about 400 μg/g of dry weight; quercetin-rhamnoside, from about 1100 to about 1300 μg/g of dry weight; quercetin, from about 100 to about 200 μg/g of dry weight; and rutin, from about 200 to about 300 μg/g of dry weight.

### Phlorizin

The apple peel polyphenolic extract of the present invention may also comprise phlorizin (also referred to as phloridzin; chemical name phloretin-2′-β-D-glucopyranoside). Phlorizin is a glucoside of phloretin, a dihydrochalcone, a family of bicyclic flavonoids, which in turn is a subgroup in the diverse phenylpropanoid synthesis pathway in plants.

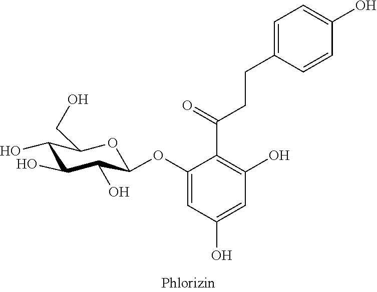

The phlorizin may be present in the apple peel polyphenolic extract of the present invention from about 1200 to about 1400 μg/g of dry weight.

### Phenolic Acids

Phenolic acids or phenolcarboxylic acids are types of aromatic acid compound. Included in that class are substances containing a phenolic ring and an organic carboxylic acid function (C₁-C₆ skeleton).

The total phenolic acid content may be from about 2000 to about 2400 μg/g of dry weight. The total phenolic acid content may comprise chlorogenic acid, from about 2000 to about 2100 μg/g of dry weight; coumaroyl glucoside, from about 10 to about 20 μg/g of dry weight; a dihydrobenzoic acid, from about 25 to about 45 μg/g of dry weight; caffeic acid, from about 1 to about 10 μg/g of dry weight; and coumaroyl hexose, from about 100 to about 200 μg/g of dry weight.

The dihydrobenzoic acid may be any one of 2,3-Dihydroxybenzoic acid (2-Pyrocatechuic acid or hypogallic acid); 2,4-Dihydroxybenzoic acid (β-Resorcylic acid); 2,5-Dihydroxybenzoic acid (Gentisic acid); 2,6-Dihydroxybenzoic acid (γ-Resorcylic acid); 3,4-Dihydroxybenzoic acid (Protocatechuic acid); 3,5-Dihydroxybenzoic acid (α-Resorcylic acid), or a combination thereof.

According to an embodiment, the apple peel polyphenolic extract of the present invention may comprise:
a proanthocyanidin (PAC) content of at least 15000 μg/g of dry weight, comprising
an about 30% to about 35% epicatechin content comprising
epicatechin monomers and oligomers having from about 1 to 5 epicatechin units, from about 3500 to 4000 μg/g of dry weight;
epicatechin oligomers having from about 6 to 10 epicatechin units, from about 1000 to 1100 μg/g of dry weight; and
epicatechin polymers having >10 epicatechin units, from about 500 to 600 μg/g of dry weight;
a total flavonol content of from about 5000 to about 5400 μg/g of dry weight; and
a total phenolic acid content of from about 2000 to about 2400 μg/g of dry weight.

According to another embodiment, there is disclosed a pharmaceutical composition comprising the apple peel polyphenolic extract of the present invention, and a pharmaceutically acceptable carrier.

Such compositions comprise a therapeutically effective amount of the apple peel polyphenolic extract of the present invention and may also include a pharmaceutically acceptable carrier.

In accordance with a method or use of the present invention compositions comprising the apple peel polyphenolic extract of the present invention may be administered to the patient by any suitable route. For example, they may be introduced into the patient by an oral, intravenous, subcutaneous, intraperitoneal, intrathecal, intravesical, intradermal, intramuscular, or intralymphatic routes, alone or as combination. The composition may be in solution, tablet, aerosol, or multi-phase formulation forms. Liposomes, long-circulating liposomes, immunoliposomes, biodegradable microspheres, micelles, or the like may also be used as a carrier, vehicle, or delivery system. The invention should not be limited to any particular method of introducing the apple peel polyphenolic extract of the present invention into the patient.

In a preferred embodiment, the composition is formulated in accordance with routine procedures as a pharmaceutical composition adapted for administration to human beings. Typically, compositions for oral administration are tablets or capsules. Typically, compositions for intravenous administration are solutions in sterile isotonic aqueous buffer. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions of the present invention have in vitro and in vivo therapeutic utilities. For example, the apple peel polyphenolic extract of the present invention can be administered to cells in culture, e.g., in vitro or ex vivo, or in a subject, e.g., in vivo, to treat non-alcoholic fatty liver disease, insulin resistance, and/or oxidative stress and inflammation. As used herein, the term “subject” is intended to include human and non-human animals. A preferred subject is a human patient with any one of non-alcoholic fatty liver disease, insulin resistance, mitochondrial dysfunction, intestinal endothelial tissue injury and/or oxidative stress and inflammation. As used herein the terms “treat”, “treating” and “treatment” of non-alcoholic fatty liver disease, insulin resistance, mitochondrial dysfunction, intestinal endothelial tissue injury and/or oxidative stress and inflammation includes: preventing the appearance of the conditions in a patient, inhibiting the onset of the conditions in a patient; eliminating or reducing a preexisting non-alcoholic fatty liver disease, mitochondrial dysfunction, intestinal endothelial tissue injury, insulin resistance, and/or oxidative stress and inflammation in a patient.

### Utilities of the Apple Peel Polyphenolic Extract

The pathogenesis of NAFLD is complex and multifactorial and the interplay of multiple physiological processes lead to steatosis and NASH. Although various physiological pathways have been identified, the list is not complete as the etiology is still enigmatic and the knowledge of the disease is progressing.

A hallmark in the development of NAFLD is the accumulation of fat in the liver—hepatic steatosis. Factors that are known to contribute to this accumulation include: (1) high free fatty acids (FFA) supply due to increased lipolysis from visceral and subcutaneous adipose tissue and dietary intake, (2) low FFA oxidation in relation to the supply of FFAs, (3) high hepatic lipogenesis, and (4) low hepatic excretion of very low-density lipoprotein (VLDL). Another hallmark is chronic inflammation causing fibrosis. Underlying processes include oxidative stress and lipid peroxidation, mitochondrial dysfunction, adipocytokine/cytokine imbalance, gut-derived bacterial endotoxins, hepatic stellate cell activation, and genetic factors. In addition, activation of Kupffer cells by cholesterol crystals is suggested to be a trigger for hepatic inflammation. Insulin resistance plays an important role in the development of both steatosis and inflammation. Because of insulin resistance, lipolysis is not inhibited by insulin. The release of FFAs cause inflammation, promote ectopic fat deposition, and further enhance insulin resistance, creating a self-propelling feed-forward process. Furthermore, insulin resistance stimulates gluconeogenesis in hepatocytes and reduces glycogen formation. Increased glucose and insulin levels stimulate de novo lipogenesis via hepatic transcription factors such as sterol regulatory element-binding protein-1c (SREBP-1c) and carbohydrate response element-binding protein (ChREBP). This causes stimulation of lipogenic enzymes such as glucokinase (gk), fatty acid synthase (FAS), and acetyl-coenzyme A carboxylase (ACC).

In the multifactorial etiology of NAFLD or NASH, oxidative stress represents a crucial process. The production of reactive oxygen species (ROS) is not balanced by the protection against ROS by antioxidants. Various potential sources of oxidative stress have been reported in NAFLD. ROS are produced during the mitochondrial and peroxisomal beta oxidation of FFAs and during the metabolism of FFAs by cytochrome P450 2E1 and 4A. ROS cause endoplasmic reticulum (ER) stress, which further promotes the accumulation of ROS within the cell. In addition, reduction in antioxidant defenses will contribute to oxidative stress. Reduced glutathione (GSH) levels and decreased superoxide dismutase (SOD), glutathione peroxidase (GPx), catalase, and glutathione transferase activities are found in NASH and appear to be correlated to disease severity. ROS react with biological compounds, including fatty acids, proteins, and DNA, causing lipid peroxidation, mitochondrial dysfunction, stellate cell activation, inflammation (via NF-kB activation), and apoptosis. Mitochondrial dysfunction and inflammation will lead to the formation of more ROS, further fueling the self-propelling feed-forward process. Iron has also been implicated in the pathogenesis of NAFLD. In patients with NAFLD, elevated hepatic iron levels have been found. The precise role of iron in the pathogenesis of NAFLD has not yet been established, but it is documented that iron increases oxidative stress, e.g., by its ability to generate hydroxyl radicals via the Fenton reaction. Citrate, an intermediate product of lipid metabolism found to be elevated in NAFLD patients, has the ability to further increase this iron-induced oxidative stress by the stimulation of the Fenton reaction.

Therefore, without wishing to be bound by theory, but in light of above, it is suggested that the prevention of the NAFLD and its treatment may implicate mechanisms involving:
Significant reduction of the oxidative stress and the inflammation;
Inhibition of de novo lipogenesis and β-oxidation of the free fatty acis (FFA) to anticipate their accumulation in the liver;
Reduction of the insulin-resistance (IR);
Prevent intestinal endothelial tissue injury;
Improvement of the gut microbiote through the selection and the proliferation of good microorganisms.

Polyphenols are members of a large family of plant-derived compounds classified as flavonoids and non-flavonoids. Non-flavonoids include stilbenes and phenolic acids. Polyphenols act as antioxidants, reducing liver fat accumulation, mainly by inhibiting lipogenesis. Epidemiological studies and meta-analyses have suggested that diets rich in plant polyphenols offer protection against cardiovascular and neurodegenerative diseases, diabetes, osteoporosis and cancer. Polyphenols may present hepatoprotective effects because they are thought to increase fatty acid oxidation, and decrease oxidative stress, insulin resistance and inflammation, the main factors responsible for the progression from NAFLD to NASH.

Liver fatty acids may also be esterified to TGs and then stored within hepatocytes or secreted into the blood as VLDL. Metabolism of fatty acids plays a key role in the development of hepatic steatosis. Liver lipid accumulation is, therefore, the result of prolonged positive energy balance (de novo lipogenesis is the preferred mechanism to stock excess energy), adipose tissue dysfunction and insulin resistance, defects in fatty acid oxidation and mitochondrial metabolism, or imbalances in lipoprotein trafficking. Hepatic TG accumulation (i.e., steatosis) is therefore one of the phenomena at the basis for hepatocyte damage that leads to a variety of other phenomenon, such as cytokines, adipokines, bacterial endotoxins, mitochondrial dysfunction and/or endoplasmic reticulum stress. It now appears that the pathophysiological etiology of NAFLD comprises several different parallel hits, represented by insulin resistance, oxidative stress, genetic and epigenetic mechanisms, cytokines and microbiota modifications, along with environmental elements. As mitochondria are an important source of ROS, redox-active compounds can be targeted to these organelles to counteract ROS production and its associated oxidative damage. However, as antioxidants, polyphenolic compounds may act directly by scavenging reactive species; and alternatively they may act indirectly by controlling the redox environment.

According to another embodiment, there is disclosed a method of preventing or treating oxidative stress, inflammation and mitochondrial dysfunction of a liver comprising administering to a subject in need thereof a therapeutically effective amount of an apple peel polyphenolic extract or a pharmaceutical composition according to the present invention.

According to another embodiment, there is disclosed a method of inhibition of de novo lipogenesis and β-oxidation of free fatty acids for preventing accumulation thereof in a liver comprising administering to a subject in need thereof a therapeutically effective amount of an apple peel polyphenolic extract or a pharmaceutical composition according to the present invention.

According to another embodiment, there is disclosed a method of preventing or treating insulin resistance comprising administering to a subject in need thereof a therapeutically effective amount of an apple peel polyphenolic extract or a pharmaceutical composition according to the present invention.

According to another embodiment, there is disclosed a method of preventing or treating intestinal endothelial tissue injury comprising administering to a subject in need thereof a therapeutically effective amount of an apple peel polyphenolic extract or a pharmaceutical composition according to the present invention.

According to another embodiment, there is disclosed a method of preventing or treating any one of oxidative stress, inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, or combinations thereof, comprising administering to a subject in need thereof a therapeutically effective amount of an apple peel polyphenolic extract or a pharmaceutical composition according to the present invention.

According to another embodiment, there is disclosed a method of preventing or treating any one of non-alcoholic fatty liver disease, non-alcoholic steatohepatitis (NASH), liver fibrosis, liver cirrhosis, and combinations thereof comprising administering to a subject in need thereof a therapeutically effective amount of an apple peel polyphenolic extract or a pharmaceutical composition according to the present invention. The non-alcoholic fatty liver disease, non-alcoholic steatohepatitis (NASH), liver fibrosis, and liver cirrhosis may comprises steatosis.

When used for therapy of any one of oxidative stress, inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, of non-alcoholic fatty liver disease, non-alcoholic steatohepatitis (NASH), liver fibrosis, liver cirrhosis; and combinations thereof, the apple peel polyphenolic extract used in the invention is administered to the patient in therapeutically effective amounts (i.e. amounts needed to treat clinically apparent conditions or diseases), or prevent the appearance of clinically apparent conditions or diseases. The apple peel polyphenolic extract used in the invention and the pharmaceutical compositions containing it will normally be administered orally or even parenterally, or intravenously.

In the method of the present invention, the therapeutically effective amount is of DAPP may be from about 100 mg/kg to about 500 mg/kg, or from about 200 mg/kg to about 400 mg/kg. According to embodiments, a low dose may be provided for prevention of diseases or conditions, and higher doses may be provided for treatment of diseases or conditions. For example, 100 mg/kg or 200 mg/kg may be provided for prevention and 400 mg/kg or 500 mg/kg for treatment of diseases or conditions.

According to an embodiment, the apple peel polyphenolic extract may be for once daily use, twice daily use, thrice daily use, or as many times a day as desired or required.

According to another embodiment, there is disclosed the use of an apple peel polyphenolic extract or a pharmaceutical composition according to the present invention, for the prevention or treatment of oxidative stress, inflammation and mitochondrial dysfunction of a liver in a subject in need thereof.

According to another embodiment, there is disclosed the use of an apple peel polyphenolic extract or a pharmaceutical composition according to the present invention, for inhibition of de novo lipogenesis and β-oxidation of free fatty acids for preventing fat accumulation thereof in a liver in a subject in need thereof.

According to another embodiment, there is disclosed the use of an apple peel polyphenolic extract or a pharmaceutical composition according to the present invention, for the prevention or treatment of insulin resistance in a subject in need thereof.

According to another embodiment, there is disclosed the use of an apple peel polyphenolic extract or a pharmaceutical composition according to the present invention, for the prevention or treatment of intestinal endothelial tissue injury in a subject in need thereof.

According to another embodiment, there is disclosed the use of an apple peel polyphenolic extract or a pharmaceutical composition according to the present invention, for the prevention or treatment of oxidative stress, inflammation and mitochondrial dysfunction of a liver; inhibition of de novo lipogenesis and β-oxidation of free fatty acids for preventing fat accumulation thereof in a liver; preventing or treating insulin resistance; preventing or treating intestinal endothelial tissue injury, or combinations thereof, in a subject in need thereof.

According to another embodiment, there is disclosed the use of an apple peel polyphenolic extract or a pharmaceutical composition according to the present invention, for the prevention or treatment of any one of non-alcoholic fatty liver disease, non-alcoholic steatohepatitis (NASH), liver fibrosis, liver cirrhosis, and combinations thereof in a subject in need thereof. The non-alcoholic fatty liver disease, non-alcoholic steatohepatitis (NASH), liver fibrosis, and liver cirrhosis may comprises steatosis.

According to another embodiment, there is disclosed the apple peel polyphenolic extract or the pharmaceutical composition according to the present invention, for use in the prevention or treatment of oxidative stress, inflammation and mitochondrial dysfunction of a liver in a subject in need thereof.

According to another embodiment, there is disclosed the apple peel polyphenolic extract or the pharmaceutical composition according to the present invention, for use in inhibition of de novo lipogenesis and β-oxidation of free fatty acids for preventing accumulation thereof in a liver in a subject in need thereof.

According to another embodiment, there is disclosed the apple peel polyphenolic extract or the pharmaceutical composition according to the present invention, for use in the prevention or treatment of insulin resistance in a subject in need thereof.

According to another embodiment, there is disclosed the apple peel polyphenolic extract or the pharmaceutical composition according to the present invention, for use in the prevention or treatment of intestinal endothelial tissue injury in a subject in need thereof.

According to another embodiment, there is disclosed the apple peel polyphenolic extract or the pharmaceutical composition according to the present invention, for use in the prevention or treatment of oxidative stress, inflammation and mitochondrial dysfunction of a liver; inhibition of de novo lipogenesis and β-oxidation of free fatty acids for preventing accumulation thereof in a liver; preventing or treating insulin resistance; preventing or treating intestinal endothelial tissue injury, or combinations thereof, in a subject in need thereof.

According to another embodiment, there is disclosed the apple peel polyphenolic extract or the pharmaceutical composition according to the present invention, for use in the prevention or treatment of any one of non-alcoholic fatty liver disease, non-alcoholic steatohepatitis (NASH), liver fibrosis, liver cirrhosis, and combinations thereof in a subject in need thereof. The non-alcoholic fatty liver disease, non-alcoholic steatohepatitis (NASH), liver fibrosis, and liver cirrhosis may comprises steatosis.

The use may be in therapeutically effective amount of from about 100 mg/kg to about 500 mg/kg or from about 200 mg/kg to about 400 mg/kg. According to embodiments, a low dose may be provided for prevention of diseases or conditions, and higher doses may be provided for treatment of diseases or conditions. For example, 100 mg/kg or 200 mg/kg may be provided for prevention and 400 mg/kg or 500 mg/kg for treatment of diseases or conditions.

The use may be once daily, twice daily, thrice daily, or as many times a day as desired or required.

According to yet another embodiment, the present invention also encompasses kits for use for prevention or treatment of non-alcoholic fatty liver disease, insulin resistance, and/or oxidative stress and inflammation in a subject in need thereof. The kits may comprise an apple peel polyphenolic extract or a pharmaceutical composition according to the present invention, and instructions on how to use the kit.

### Extraction Process

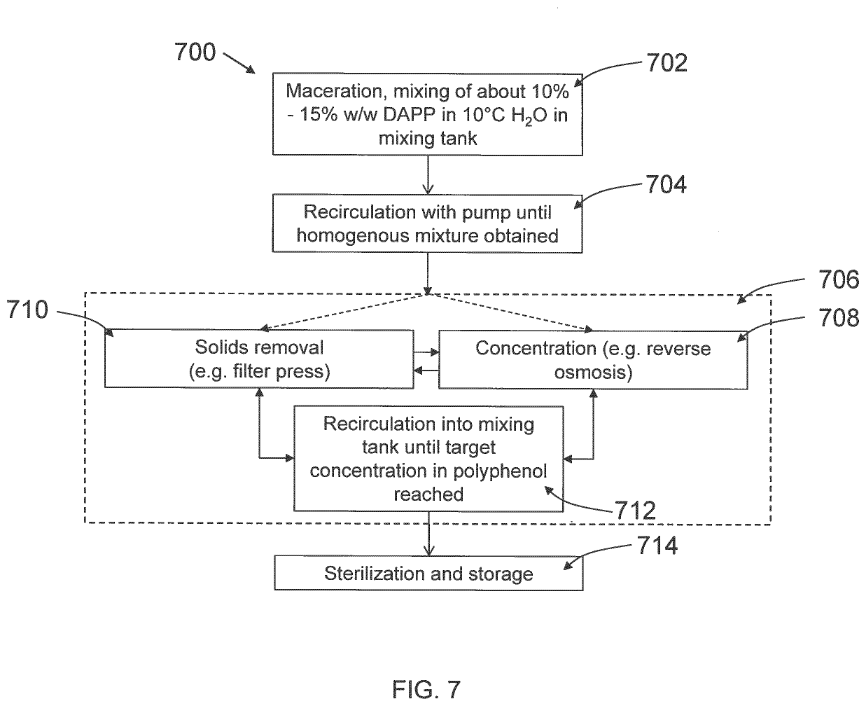

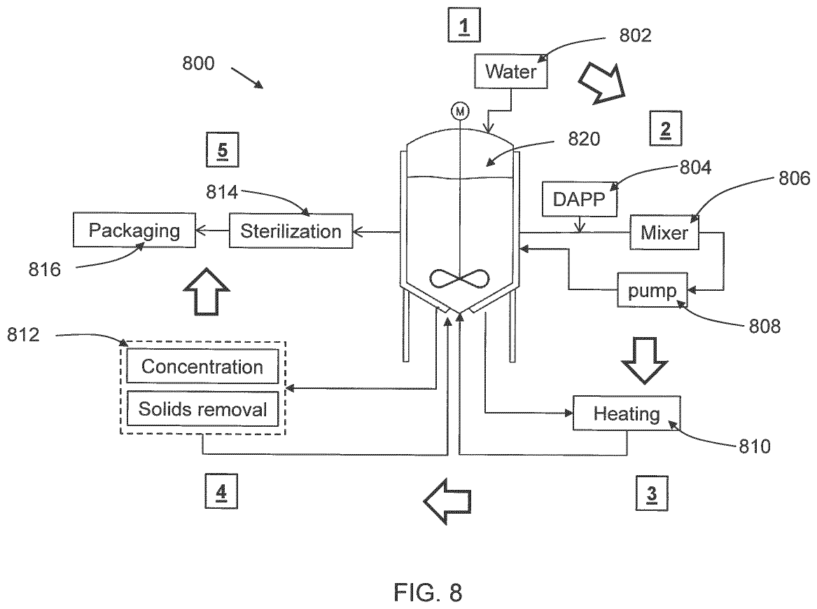

Now referring to FIGS. 7 and 8. According to another embodiment, there is disclosed a process for extraction of at least a polyphenolic content from dry apple peel powder (DAPP). The process is a gentle, cold water-based process that does not involve the use of solvents other than water, does not involve the use of enzymes to digest the DAPP material. The process may be performed in the absence of light, to avoid light-induced degradation of the polyphenolic content.

The process comprises the steps of macerating a first mixture comprising from about 10% to about 15% w/w DAPP, and water at about 8° C. to about 12° C., for a time sufficient and with sufficient mechanical energy to obtain a homogeneous mixture and extract a soluble polyphenol content from the DAPP. This is followed by a step of heating the first mixture to a temperature of about 48° C. to about 52° C., or preferably 50° C., to obtain a second mixture. Next, the second mixture is subjected to at least one step of concentration comprising 1) concentrating the second mixture for concentration of a soluble fraction therefrom, 2) filtering the second mixture for removal of a solid fraction therefrom, or 3) a combination thereof, to obtain a concentrated polyphenol fraction. The concentration step is performed until the desired concentration of extracted polyphenol is reached. In embodiment, the desired concentration may be from about 20% w/w to about 40% w/w.

Referring to FIGS. 7 and 8, in the process 700 at step 702, the 10%-15% w/w DAPP (804) is mixed with the water (802) at about 8° C. to about 12° C., preferably at a temperature of 10° C., and macerated with sufficient agitation provided by means such as a sheer mixer (806), before the first mixture obtained therefrom is reintroduced (e.g. through pump 808) in mixing tank (820) until an homogeneous mixture of the DAPP and water is obtained, to extract the soluble polyphenol present in the DAPP. According to an embodiment, the DAPP may be DAPP having an about 100 mesh size (approximately 149 microns), or from about 100 mesh to about 200 mesh (approximately 149 microns to about 74 microns).

After sufficient maceration and mixing, when the first mixture is sufficiently homogenous, the first mixture is heated (810) to a temperature of about 48° C. to about 52° C., preferably 50° C., to obtain a second mixture.

The second mixture comprises a soluble fraction which contains the polyphenol content and a solid fraction which contains the solid residues from the DAPP.

Next, pursuant to the heating, the second mixture is subjected to at least one, and usually a plurality of step of concentration, referred to as 706 in FIG. 7. The goal of the concentration step is to remove the DAPP residual solids, as well as the excess water introduced to maximally extract the polyphenol from DAPP. This step comprises concentrating the second mixture for concentration of a soluble fraction therefrom (708/814), filtering the second mixture for removal of a solid fraction therefrom (710/812), to obtain a concentrated polyphenol fraction (714). The concentration step is performed until the desired concentration of extracted polyphenol is reached, for example between about 20% w/w to about 40% w/w and then the concentrated polyphenol fraction is cooled to a temperature of about 6° C. to about 10° C., preferably 8° C. Therefore, the concentration step (706) may be performed once, or it may be performed a plurality of times, until the desired concentration of polyphenol is reached, and then the concentrated polyphenol fraction is cooled to a temperature of about 6° C. to about 10° C., preferably 8° C. According to other embodiments, the solids removal step (710 /812) may be performed first, followed by the concentration step (708/812) second. According to an embodiment, the concentration step (710/812) may be performed first, followed by the solids removal step (710/812). According to another embodiment, the solids removal step (710) and the concentration step (708) may be performed on different portions of the second mixture, which are later combined and retreated until the desired concentration of polyphenol is reached, and then the concentrated polyphenol fraction is cooled to a temperature of about 6° C. to about 10° C., preferably 8° C.

According to some embodiments, the filtering may be performed with a filter having a pore size of from about 2 μm to about 45 μm, or from about 5 μm to about 45 μm, or from about 10 μm to about 45 μm, from about 20 μm to about 45 μm, from about 30 μm to about 45 μm, or from about 5 μm to about 30 μm, or from about 10 μm to about 30 μm, from about 20 μm to about 30 μm, or from about 5 μm to about 20 μm, or from about 10 μm to about 20 μm, or from about 5 μm to about 10 μm, with a solid support filter, with a filter press, by centrifugation, or combination thereof. The solid support filter may be for example a diatomaceous earth filter. According to some embodiments, the concentrating may be performed with a concentrator unit. For example, the concentrator unit may be a reverse osmosis unit, a vacuum evaporation unit, or a combination thereof.

Pursuant to the concentration step, the concentrated polyphenol fraction may be sterilized or pasteurized (714/814) to obtain a sterilized (or pasteurized) concentrated polyphenol fraction, and packaged (714/816). According to embodiments, the sterilization or pasteurization may be performed with any suitable known sterilization or pasteurization technique, such as high pressure pasteurization (HPP or Pascalization), UV light treatment, pulsed electric field (PEF), or combination thereof, for example, followed by freezing of the product. According to another embodiment, a step of microfiltration may be performed prior to the step of sterilizing/pasteurizing, for removal of remaining small particles in the concentrated of polyphenol is reached.

In use, the concentrated polyphenol fraction may be further concentrated using known concentration technologies in order to further increase concentration so as to facilitate use and/or consumption of the product.

The present invention will be more readily understood by referring to the following examples which are given to illustrate the invention rather than to limit its scope.

### Example 1

### Preparation of DAPP Concentrated Polyphenol Fraction

A volume of 1360 liters (300 gallons) of water at temperature of 10° C. was added to a mixing tank 820 with 160 kilograms of dried apple peel powder (DAPP) at 100 mesh. The mixture was left for maceration for few minutes then rotated slowly into the tank. Then, a zeolite based powder was added for filtration purpose of the mixture along with water and pumped through a starch pump 810 while the system is circulating from and back to the mixing tank. The mixture was again left until the mixture temperature went up to 18° C. Then the mixture was diverted in a first part towards filter press 812 and a second part to reverse osmosis apparatus 814 and back to mixing tank 820. The process was pursued until the concentration/reduction of the initial volume down to 275 liters of aqueous mixture. The remaining volume of 275 liters left is then sent to a sterilization unit 816 where the volume is treated with either high pressure pasteurization, UV treated, or combination thereof, followed by freezing, and packaged (818). Afterward, the concentrated extract for further subjected to concentration until desired concentration is obtained.

PAC isomers and other anthocyanidins, quercetin flavonol polymers, phlorizin, phenolic acid compounds, total triterpenoids and acid and pigments were determined as follows:

Procyanidins were analyzed by normal-phase analytical HPLC using an Agilent® 1260/1290 Infinity™ system equipped with a fluorescence detector. The separation was performed at 35° C. with a flow rate of 0.8 ml/min using a Develosil Diol™ column (250 mm×4.6 mm, 5 μm particle size), protected with a Cyano SecurityGuard™ column (Phenomenex™). The elution was performed using a solvent system comprising solvents A (acetonitrile/acetic acid 98/2 v/v) and B (methanol/ultrapure water/acetic acid 95/3/2 v/v/v) mixed using a linear gradient from 0% to 40% B for 35 min, 40% to 100% B for 5 min, 100% isocratic B over 5 min and 100% to 0% B for 5 min. The fluorescence was monitored at excitation and emission wavelengths of 230 and 321 nm. Procyanidins with degrees of polymerization (DP) from 1 to >10 were quantified using external calibration curve of epicatechin, taking into account their relative response factors in fluorescence.

Phenolic acids, flavonols and flavan-3-ols were characterized using a Waters® Acquity™ UPLC-MS/MS equipped with an H-Class quaternary pump system, a flow through needle (FTN) sample manager system and a column manager. The MS detector was a TQD mass spectrometer equipped with a Z-spray electrospray interface. The analysis was achieved using an Agilent® Plus™ C18 column (2.1 mm×100 mm, 1.8 μm). The separation was performed at 40° C. at a flow rate of 0.4 ml/min with a mobile phase consisting of 0.1% formic acid in ultrapure water and acetonitrile (solvent A and B respectively) using a gradient elution as follows: 0-4.5 min, 5-20% B; 4.5-6.45 min, 20% B; 6.45-13.5 min, 20-45% B; 13.5-16.5 min, 45-100% B; 16.5-19.5 min, 100% B; 19.5-19.52 min, 100-5% B; 19.52-22.5 min, 5% B. The MS/MS analyses were carried out in negative mode using electrospray source parameters as follows: electrospray capillary voltage was 2.5 kV, source temperature was 140° C., desolvation temperature was 350° C., cone and desolvation gas flows were 80 L/h and 900 L/h respectively. Data were acquired through multiple reaction monitoring (MRM) using Waters® Masslynx™ V4.1 software. Phenolic standards were analyzed using the same parameters and used for the quantification, when available. Otherwise, the phenolic compounds were quantified using their aglycone or the most similar phenolic structure. Dry samples were prepared as follows: 50 mg diluted in AWA (acetone/water/acetic acid (70:29.5:0.5) v/v) filtered on 0.2 μm nylon filter and diluted two times for accurate measurement. Liquid samples were prepared as follows: diluted in AWA (acetone/water/acetic acid (70:29.5:0.5) v/v) filtered on 0.2 μm nylon filter and diluted two times for accurate measurement.

The MRM transitions for phenolic acids, flavonols and flavan-3-ols are shown in Table 1 below:

TABLE 1
MRM transitions

| | MRM | |
|---|---|---|
| Flavonols | | |
| [symbol] | 447 | [symbol] 285 |
| [symbol] | 301 | [symbol] 150 |
| [symbol] | 40 [symbol] | [symbol] 301 |
| [symbol] | 433 | [symbol] 301 |
| [symbol] | 609 | [symbol] 301 |
| [symbol] | 433 | [symbol] 301 |
| [symbol] | 4 [symbol] | [symbol] 301 |
| [symbol] | 447 | [symbol] 301 |
| [symbol] | 625 | [symbol] 301 |
| [symbol] | 315 | [symbol] 151 |
| [symbol] | 317 | [symbol] 151 |
| [symbol] | 479 | [symbol] 317 |
| Flavan-3-ols | | |
| [symbol] | 289 | [symbol] 245 |
| [symbol] | 289 | [symbol] 245 |
| Phenolic acids | | |
| [symbol] acid | 137 | [symbol] 93 |
| [symbol] acid | 153 | [symbol] 109 |
| [symbol] acid | 1 [symbol] 9 | [symbol] 125 |
| [symbol] acid | 163 | [symbol] 114 |
| [symbol] acid | 163 | [symbol] 119 |
| [symbol] acid | 179 | [symbol] 135 |
| [symbol] acid | 193 | [symbol] 1 [symbol] 4 |
| [symbol] | 325 | [symbol] 163 |
| [symbol] | 341 | [symbol] 179 |
| [symbol] acid | 173 | [symbol] 93 |
| [symbol] acid | 301 | [symbol] 145 |
| 3 [symbol] acid | 353 | [symbol] 191 |
| 4 [symbol] acid | 353 | [symbol] 191 |
| 5 [symbol] acid | 353 | [symbol] 191 |

[symbol]

indicates data missing or illegible when filed

### Example 2

### Research Design and Methods

Animals

The animal model used in the present study was the Psammomys. obesus, a rodent model that exhibits genetic predisposition to CMD [25-28,30,31,33-35] and, in particular, NAFLD [27,30,39,40]. The experimental protocol was similar to that of previous studies [26,28,31] except for the addition of DAPP. DAPP has been prepared following the process described herein in Example 1. Briefly, following weaning, the animals were randomly divided into three groups: the first one was assigned to a low-energy diet (LE, 2.38 kcal/g), the second to a high-energy diet (HE, 2.93 kcal/g) capable of inducing IR, and the third to the same HE diet but with a daily dose of 200 mg/kg DAPP by gavage (HE+DAPP), whereas the other two former groups received the vehicle (water). Importantly, our previous studies revealed that flavonoids figure among the major polyphenol components of DAPP, while flavonols constituted the dominant subclass present as a mixture of aglycone and glycosylated quercetin and dihydrochalcone.

Weight and tail capillary glucose were measured weekly in all animals. At the end of the 8-week experimental period, feces were collected, the animals were sacrificed, and plasma samples were collected from inferior vena cava. Animal experiments were carried out in accordance with the guidelines of the Institutional Committee for Care and Use of Laboratory Animals.

In Vivo Intestinal Fat Absorption

One week before sacrifice, mice were subjected to a fat meal in order to examine the impact of DAPP on chylomicron (CM) output. A volume of 2 ml of 2% intralipid was orally administered by gavage in 8-h fasted animals. One hour later, Triton WR-1339 (400 mg/kg body weight) was injected to inhibit CM clearance by lipoprotein lipase as described previously [28]. Arterial blood samples were collected 0, 120 and 240 min after Triton administration. CMs were isolated by ultracentrifugation as described previously [31]. Plasma triglycerides (TG) and CM-TG were quantified enzymatically.

In Vitro Chylomicron Assembly and Secretion

The organ culture technique was employed to assess the various steps governing CM formation as described in [25,28,31,41-49]. Briefly, the jejunum from P. obesus was washed and cut into explants, which were transferred onto lens paper, with the mucosal side facing up in each organ culture dish. After a 30-minute stabilization period, the medium was replaced with a fresh one containing a final amount of 1.0 μmol/mL unlabeled oleic acid with 0.5 μCi of [¹⁴C]-oleic acid in a micellar mixture (6.6 mM sodium taurocholate, 1 mM oleic acid, 0.5 mM monoolein, 0.1 mM cholesterol, and 0.6 mM phosphatidylcholine) [28,31]. Intestinal explants from P. obesus were cultured for 2 hours. After this incubation period, tissue integrity was confirmed by morphologic (lighted electron microscopy) and biochemical studies (sucrase activity, villin expression).

For the determination of secreted CM, the medium supplemented with anti-proteases was first mixed with a plasma lipid carrier (2:0.6 vol/vol) to efficiently isolate the newly synthesised TG-rich lipoproteins. The latter were then isolated at a density of 1.006 g/ml by spinning at 100,000×g for 2.26 h with a tabletop ultracentrifuge (Beckman Instruments) as described previously [28,31]. In some experiments, Pluronic L-81 (0.5%) was added to the HE diet one week before animal sacrifice in order to block CM synthesis.

De Novo Apolipoprotein (Apo) B-48 Synthesis

Jejunal explants with 300 μCi [³⁵S]-methionine, washed with methionine-free Leibovitz medium and homogenized in PBS containing 1% (wt/vol) Triton X-100, methionine (2 mM), phenylmethylsulfonyl fluoride (1 mM), and benzamidine (1 mM). The homogenates were centrifuged (4° C.) at 105,000×g for 60 min and supernatants subsequently reacted with excess Apo B polyclonal antibodies for 18 h at 4° C. Anti-P. obesus Apo B antiserum was raised in rabbits. Pansorbin was then added, and the mixture was reincubated at 20° C. for 60 min. The immunoprecipitates were washed extensively and analyzed by a linear 4-20% acrylamide gradient preceded by a 3% stacking gel as described previously [28,31]. Apo B-48 bands on gels were sectioned and counted after an overnight incubation at 20° C. with 1 ml BTS-450 (Beckman) and 10 ml liquid scintillation fluid (Ready Sol. NA, Beckman).

Microsomal TG Transfer Protein (MTP) Assays

Intestinal microsomes were used as the source of MTP activity that was determined by the transfer of radiolabeled TG from donor small unilamellar vesicles as described previously [31,48,50].

Monoacylglycerol Acyltransferase (MGA T) and Diacylglycerol Acyltransferase (DGAT) Activities

The activities of MGAT and DGAT were also determined in microsomes as reported previously [30,31,49].

AMPK Activity Assay

AMPK activity of liver homogenates was measured using the synthetic SAMS peptide substrate HMRSAMSGLHLVKRR (SEQ ID NO:1) phosphorylation assay [51]. Briefly, liver homogenates, containing 0.8 mM DTT and 0.2 mM AMP with or without 0.2 mM SAMS peptide, were added to the reaction mixture: 5 mM MgCl₂, 0.2 mM ATP, and [³²P]-ATP for 10 min at 37° C. Aliquots of the reaction mixture supernatant were spotted on Whatman filter paper (P81). The filters were washed with cold 150 mM phosphoric acid for 40 min and with acetone for 20 min and allowed to dry before scintillation counting. The difference between the presence and absence of [³²P] was calculated as the AMPK activity in picomoles per minute per milligram proteins.

Intestinal Permeability and Gut Membrane Integrity

Following a 6-hour fasting, the animals were given 4,000-Da fluorescein isothiocyanate (FITC)-dextran (600 mg/kg body weight, 125 mg/mL) by gavage as described previously [52,53]. Blood was collected 1 hour after the gavage and centrifuged at 4° C., 4,000 rpm, for 5 minutes. Plasma was diluted in an equal volume of PBS (pH 7.4) and analyzed for FITC-dextran concentration with a fluorescence spectrophotometer at the excitation wavelength of 485 nm and the emission wavelength of 535 nm. Standard curve for calculating the FITC-dextran concentration in the samples were obtained by diluting FITC-dextran in non-treated plasma diluted with PBS (1:2).

The content of zonulin (ZO) and occludin was measured in intestinal homogenates using ELISA kits (LifeSpan Biosciences, Inc.). Lipopolysaccharides (LPS) were assayed with an Elisa kit according to the manufacturer's instructions (TSZ Elisa, Waltham, Mass., USA). The concentrations of trimethylamine-N-oxide (TMAO), a gut microbial-dependent metabolite, was quantified in plasma and liver tissues using an LC/MS. The isocratic mobile phase was composed of 50% water and 50% acetonitrile containing 0.1% formic acid at a flow rate of 1 ml/min.

RNA Extraction and Quantitative RT-PCR

Total RNA was extracted from intestinal samples using TRIzol Reagent (Invitrogen®, Carlsbad, Calif.) according to the manufacturer's specifications, repurified, checked for integrity by agarose gel electrophoresis, and reverse transcribed into cDNA using the Superscript First Strand Synthesis System (Invitrogen). The cDNA was used as template for RT-PCR analysis. Primers against the genes of interest were designed using the available mRNA sequence information in NCBI GenBank. Basic local alignment search tool (BLAST, NCBI, available at http://www.ncbi.nlm.nih.gov/blast/Blast.cgi) was used for primer verification. Quantitative RT-PCRs were performed using Quantitect SYBR Green kit (Applied Biosystems, Foster City, Calif.) in an ABI Prism 7000 Sequence Detection System. The qRT-PCRs were carried in a 96-well plates with a final volume of 25 μl per well; 12.5 μl of SYBR Green Mix (2×) were added to a well containing 25 μmol of the forward and reverse primers and 0.5 μg of cDNA template in a total of 12.5 μl of diethylpyrocarbonate H₂O. Subsequently, negative controls without cDNA were prepared. The reaction of amplification was carried out using 40 cycles. To normalize the different cDNA sample amounts, the housekeeping gene glyceraldehyde-3-phosphate dehydrogenase (GAPDH) was employed as a reference gene. The analyses were performed in triplicate for each gene and for GAPDH in the same plate. The relative mRNA fold-changes between the animal groups were calculated using the 2^{-ΔΔ}Ct method [25-27].

Biochemical Analyses

Plasma glucose was determined by an enzymatic glucose analyser, and insulin levels were assessed by radioimmunoassay using a human primary antibody (Phadesph; Kabi Pharmacia Diagnostics, Uppsala, Sweden). Liver TG and total cholesterol (TC) levels were measured colorimetrically after lipid extraction (Boehringer Mannheim, Montreal, QC, Canada). Tissue samples were used to determine myeloperoxidase (MPO) activity. Tissue homogenates were centrifuged at 5000×g at 4° C. for 15 min and MPO activity in the supernatant was analysed by ELISA (Hycult Biotech). All the samples were analysed on the same plate at 450 nm. Estimation of lipid peroxidation was assessed by measuring the content of malondialdehyde (MDA) in tissue samples by HPLC [54] or hydrogen peroxide (H₂O₂) by commercial kit [37]. Endogenous antioxidant enzyme activity was determined as reported previously [38,55]. Commercial enzyme linked immunosorbent assay kits were used to determine the levels of ATP [37], 8-hydroxydeoxyguanosine (8-OHDG) [56,57], tumor necrosis factor alpha (TNF-α) and interleukin 1beta (IL-1β) (R&D Systems). The activities of respiratory chain complexes were assayed as previously described following isolation of mitochondria [56,57].

Statistical Analysis

Statistical analyses of data were performed with Prism 4.03 software (GraphPad Software). All values were expressed as the mean±SEM. The data were evaluated by ANOVA when appropriate, and the differences between the means were assessed using the Bonferroni's multiple comparison test. A P-value of less than 0.05 was considered to be significant.

### Example 3

### Anthropometric, Metabolic and Liver Parameters

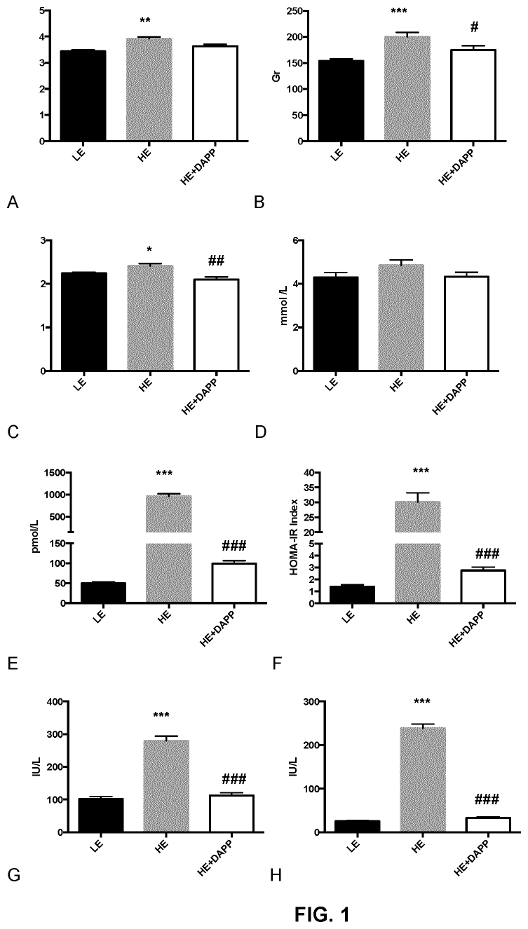

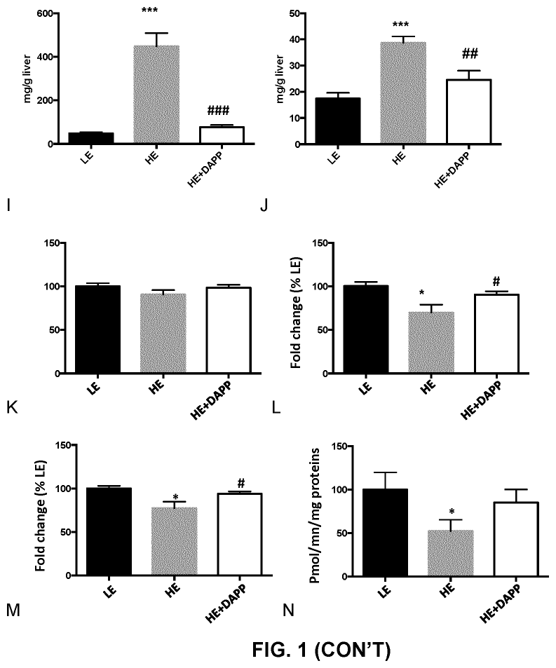

Body weight was significantly increased in rats fed a HE diet (FIG. 1A), when compared to LE rats, but this weight gain was partly reduced with DAPP treatment. However, significant changes were noted in liver weight and hepatosomatic index in the HE+DAPP group (FIGS. 1B-1D). Similarly, insulinemia and raised HOMA-IR (the IR index) in the HE group were markedly diminished by the addition of DAPP (FIGS. 1D-1F). Concomitantly, transaminases and liver lipids (TG & TC) responded to DAPP supplementation by showing a significant decline whereas the gene expression of insulin signaling factors (IRS2, GSK3β) clearly exhibited a marked elevation (FIGS. 1G-1N). From these findings, it was elucidated that DAPP caused significant improvement of systemic and hepatic insulin sensitivity along with NAFLD alleviation.

### Example 4

### Liver OxS, Antioxidant Defense and Inflammatory Levels

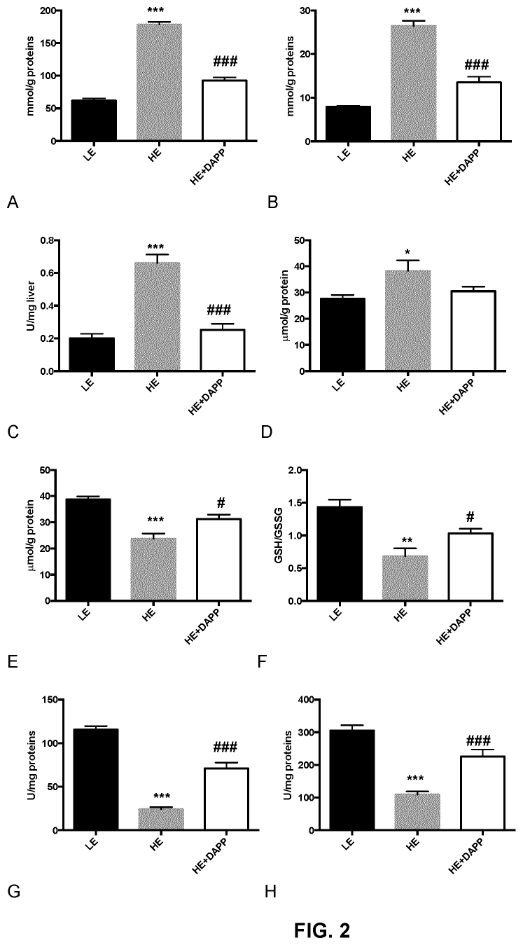

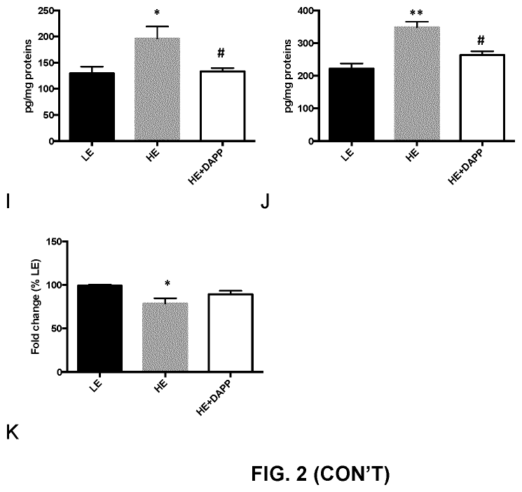

The NAFLD condition in the HE fed-animals was characterized by high levels of hepatic H₂O₂, MDA and MPO, as well as reduced antioxidant defense, reflecting OxS and lipid peroxidation (FIGS. 2A-2H) in the liver of HE animals. Additionally, HE P. obesus displayed a higher level of the key inflammatory markers TNFα and IL-1β, indicating the occurrence of inflammation (FIGS. 2I-2K). These two processes were underpinned by the declined gene expression of NRF2 transcription factor (FIG. 2L). However, DAPP supplementation alleviated OxS and inflammation while strengthening the antioxidant defense. Therefore, DAPP could efficiently protect against OxS and inflammation, thereby preventing IR and alleviating fatty liver disease in the P. obesus animal model.

### Example 5

### Intestinal Permeability, Endotoxemia and Stool Short-Chain Fatty Acids

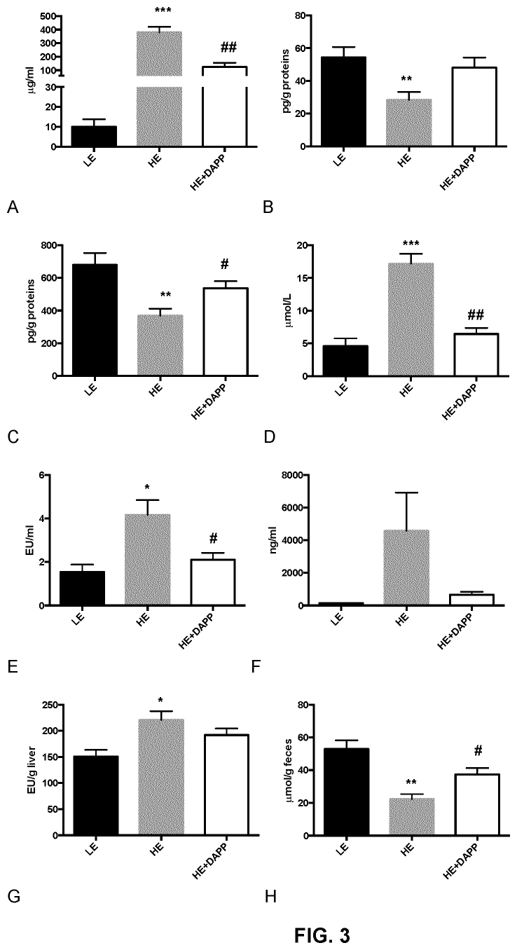

The next step was to determine whether endotoxemia resulting from intestinal hyperpermeability occurs in HE P. obesus animals, and whether these two complications are downregulated by DAPP administration. Assessment of intestinal integrity by FITC-dextran penetration showed higher plasma concentrations of FITC-dextran in HE compared to LE group, underlining mucosal barrier impairment (FIG. 3A). Accordingly, the level of the two important tight junction proteins ZO and occludin was lower in the intestine of the P. obesus animals exposed to HE (FIGS. 3B-3C). Consequently, the disruption of intestinal barrier function led to the enhanced passage of LPS and TMAO into the circulation and LPS into the liver, whereas as a drop of short chain fatty acids (SCFA) was noted in the HE group compared to P. obesus animals on LE (FIGS. 3D-3H). However, DAPP treatment decreased intestinal permeability, probably via the attempt to restore the tight junction proteins, which suggests its great capacity to defend intestinal barrier function while protecting against LPS-induced liver injury (FIG. 3).

### Example 6

### Liver Gluconeogenesis, Lipogenesis and Fatty Acid Oxidation

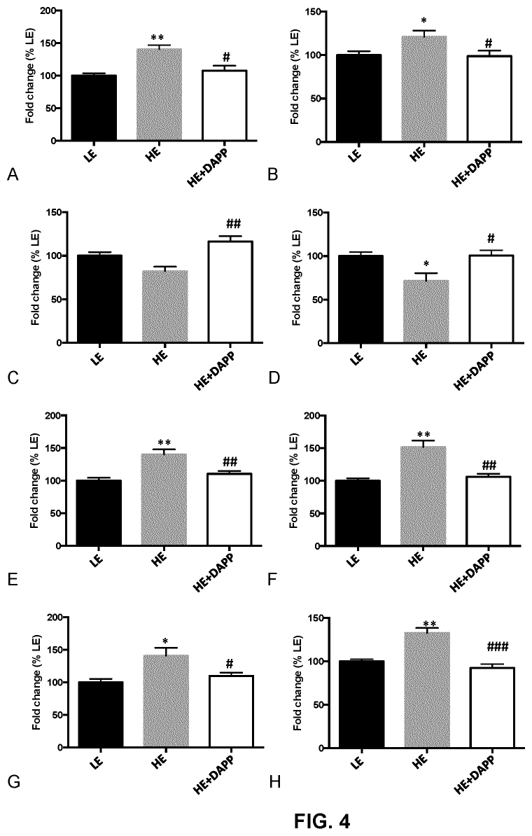

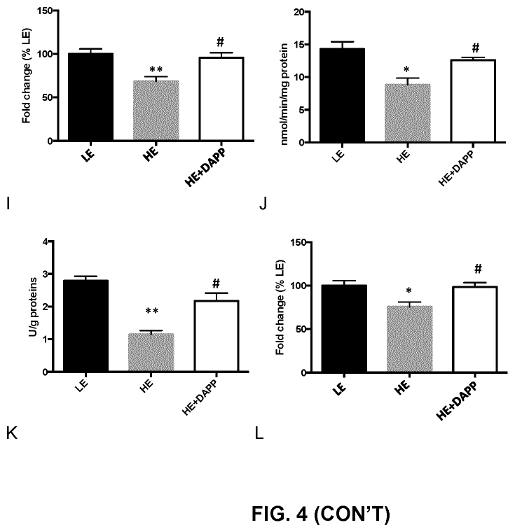

As IR directly disturbs glucose metabolism in the liver, thereby contributing to significant steatosis, it was important to determine the expression of the two critical hepatic gluconeogenic enzymes, G6Pase and PEPCK in P. obesus. While IR markedly enhanced their transcripts, DAPP were able to repress their gene expression (FIGS. 4A-4B). As PPARβ and FOXO1 transcription factors control the expression of these key enzymes, their gene expression were analyzed, which was significantly curbed by IR and heightened by DAPP (FIGS. 4C-4D).

In view of the relationship between hepatic lipid accumulation and de novo lipogenesis [58], key lipogenic enzymes ACC and FAS were evaluated, as well as PPARγ and SREBP-1c, two key transcription factors upregulating de novo lipogenesis in the liver. The findings revealed their raised mRNA expression in the HE group, which was attenuated by DAPP supplementation (FIGS. 4E-4H).

Since hepatic fatty acid (FA) oxidation may alter NAFLD pathogenesis, FA β-oxidation was analysed via its regulatory enzymes. A substantial drop was noted in the expression of CPT1α and acylCoA oxidase in association with the decreased transcription factor PPARα in the HE group. In contrast, DAPP treatment reversed the activities of FA oxidation enzymes to near the value of P. obesus on LE diet (FIGS. 4I-4L).

### Example 7

### Mitochondrial Functions

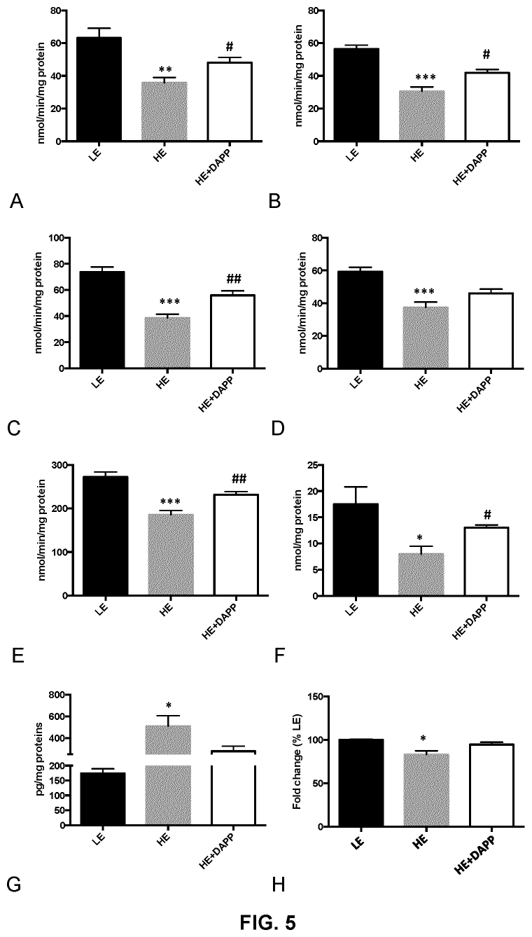

As mitochondrial dysfunction has been suggested to play a significant role in the development and progression of NAFLD [12], we assessed mitochondrial function by analyzing respiratory chain and energy homeostasis in freshly isolated organelles from liver specimens in P. obesus. Our experiments showed a noticeable decline in mitochondrial complex enzymatic activities in the liver of HE group (FIGS. 5A-5E). We then estimated the hepatic ATP homeostasis and also observed a reduction in the liver ATP content in HE animals when compared to the LE group (FIG. 5F). The concentration of 8-OHDG, a marker of oxidative DNA damage and mutagenicity, was also augmented in the mitochondria of animals on HE diet (FIG. 5G). However, DAPP treatment resulted in a significant amelioration of mitochondrial dysfunction parameters, including peroxisome proliferator-activated receptor-gamma co-activator-1 alpha (PGC-1α), which represents a powerful transcription factor regulating mitochondrial biogenesis, oxidative phosphorylation and energy homeostasis (FIG. 5G).

### Example 8

### In Vivo and In Vitro Intestinal Fat Transport and CM Production

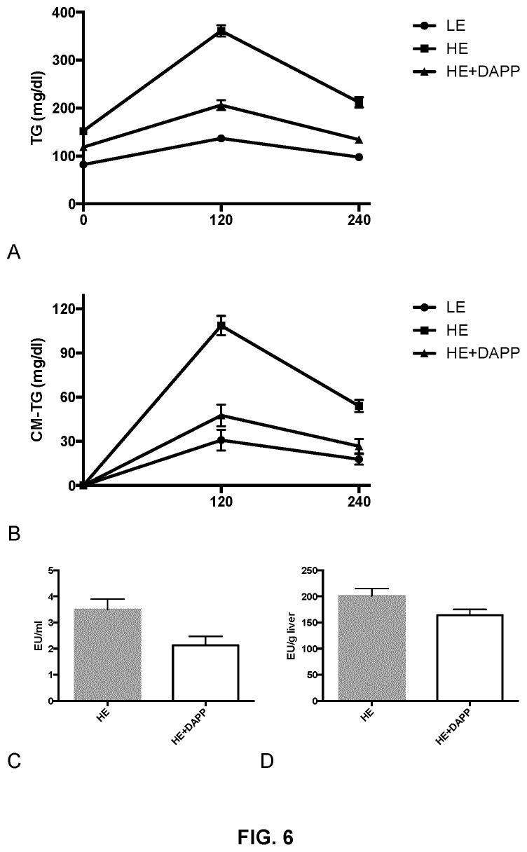

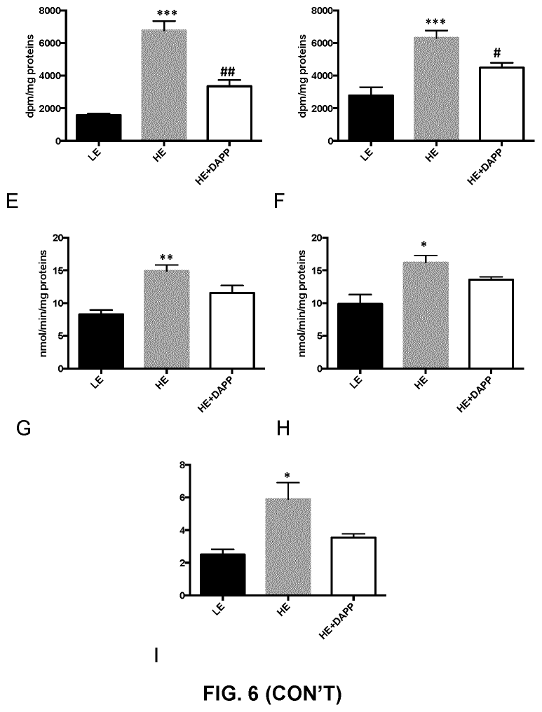

Given that gut-liver axis has been suggested as a crucial player in the pathogenesis of CMD, including NAFLD, it was hypothesized that overproduction of intestine-derived CMs occur in the IR condition in response to HE diet, which may lead to stimulated CM remnant uptake and may promote NAFLD. Therefore, we carried out experiments to evaluate intestinal fat absorption following intralipid administration by gavage in 12-h fasted P. obesus animals. The secretion of postprandial circulating TG and CM-TG was higher in the blood circulation of HE animals when compared to the LE group (FIGS. 6A-6B).

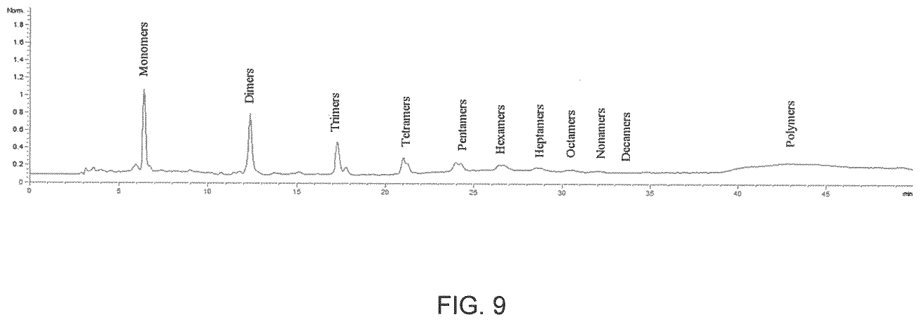

Besides their essential function in the transport of dietary lipids, CM particles stimulate intestinal absorption of LPS [59], which causes inflammation in the liver and deteriorates NAFLD [60,61]. The hypothesis that DAPP may restrain LPS transfer to the circulation and liver by limiting CM supply was tested. Overall, the data showed the ability of DAPP to lessen IR-mediated CM and liver LPS content. To determine whether HE diet-stimulated LPS secretion was related to CM formation, Pluronic L-81, a surfactant inhibiting CM synthesis, was administered to P. obesus animals on the HE diet. Clearly, impeding CM delivery to the blood circulation reduces circulating and hepatic LPS concentrations (FIGS. 6C-6D). The combination of DAPP and Pluronic L-81 only slightly lowered LPS concentrations, which suggests similar mechanisms of action, i.e. CM flow blocking-mediated LPS fall. To highlight the DAPP mechanisms of action, which inhibited CM output and concomitantly slowed down LPS transport, the culture of jejunal explants was performed. Our findings showed that DAPP abolished the overproduction of intestinal TG lipoproteins by affecting the biogenesis of Apo B-48 and the activity of MTP, MGAT and DGAT in P. obesus exposed to HE (FIGS. 6E-61).

Apple polyphenols have been reported to exert various beneficial effects on human health. However, the accurate mechanisms for their favorable impact have not been delineated so far. Moreover, little is known on their preventive/therapeutic role in CMD and particularly in NAFLD. The major aim of the present study was to investigate the capacity of DAPP to modulate several processes implicated in the pathogenesis of NAFLD, with an emphasis on the deregulation of gut-liver axis crosstalk. Our exhaustive work demonstrated the effectiveness of DAPP to (1) alleviate the severity of IR and NAFLD; (2) lessen OxS and inflammation levels, as well as mitochondrial dysfunction in the liver; (3) weaken associated molecular patterns such as intestinal permeability, inter-epithelial tight junctions and LPS translocation from the gut to the blood circulation and liver; (4) downregulate the assembly and secretion of CMs, thereby improving postprandial dyslipidemia and the transport of LPS that induce endotoxemia; and (5) enhance the output of SCFA by fighting dysbiosis.

In the present work, the P. obesus (sand rat) was employed, a well-established model of nutritionally induced obesity and CMD [25-31,34-36], which shares most metabolic parameters of the human MetS [31] and clearly exhibits features of NAFLD [26,27,30,39,40]. Indeed, similarly to humans, these animals under calorie-enriched diet develop not only obesity, IR and dyslipidemia, but also liver steatosis with intrahepatic macrovesicular fat infiltration and signs of disease progression [39,40,62]. Only insulin-resistant animals without hyperglycemia were included in the present study.

Exposure of P. obesus animals to HE diet led to the development of hyperinsulinemia, systemic IR, elevated plasma transaminases and hepatic accumulation of lipids. Their liver also displayed OxS and inflammation, two major factors that elicit local insulin insensitivity, which was confirmed in our experiments by diminished expression of IRS1, IRS2 and GS3Kβ, as well as AMPK activity. Administration of DAPP significantly lowered hepatic injury as reflected by a substantial decline in steatosis, transaminases, OxS and inflammation. These beneficial effects were accompanied by a drop in systemic and hepatic IR, which constitutes an invariable accompaniment of NAFLD and is largely recognized as the most common underlying risk factor for the development of not only type 2 diabetes mellitus but also life-threatening diseases such as NASH, liver cirrhosis, hepatocellular carcinoma, and MetS in patients with NAFLD [63,64].

The mechanisms underlying the development of NAFLD are not completely understood but likely implicate various pathways in P. obesus, including lipogenesis, gluconeogenesis and FA β-oxidation in the liver as a result of IR. In fact, hepatic steatosis appears as a consequence of stimulated de novo lipogenesis in view of the high transcription of lipogenic genes in link with the induction of SREBP-1c and PPAR-γ, and mitigated FA β-oxidation reflected by the inhibition of critical regulatory enzymes because poor PPAR-α expression. Therefore, elevated de novo lipogenesis occurred in the liver of P. obesus without a compensatory enhancement of FA oxidation, which promoted lipid deposition. By contrast, DAPP exerted an anti-lipogenic effect by decreasing de novo lipogenesis and TG synthesis in addition to increasing FA β-oxidation, which led to a reduced lipid accumulation. This finding is likely ascribed to the effectiveness of DAPP to alter several upstream transcription regulators involved in hepatic FA metabolism.

As excess glucose is converted into lipids in the liver, gluconeogenesis in P. obesus was assessed via its regulatory enzymes. Apparently, under the IR condition, the liver produced more glucose via the actions of gluconeogenic enzymes whose expression was high. As reported by others, accumulated FAs in the liver may activate PEPCK, thereby producing glucose production, which can lead in the long run to hepatic glycemic burden [65]. Again, DAPP improved the metabolic situation by involving the powerful transcription factors PPAR-β and FOXO-1, which downregulated PEPCK and G6Pase. Another mechanism is via the stimulation of AMPK activity by DAPP, as AMPK is able to promote catabolism through the modulation of PPARs (controlling lipogenesis and FA β-oxidation) and SREBP-1c (inhibiting PPAR-γ mediated activation of lipogenic genes) while also downregulating gluconeogenesis [66-68]. Overall, these findings provide a clear line of evidence about the potential of DAPP to treat/alleviate hepatic steatosis.

Our experiments evidenced abnormalities in mitochondrial β-oxidation, respiratory chain, energy production and DNA integrity in the liver of insulin-resistant P. obesus, which confirms the assumption that mitochondrial dysfunction plays a role in the pathogenesis of NAFLD [69,70]. Without entering deeper into the complex issue whether mitochondrial dysfunction is a key pathogenic event in NAFLD or the consequence of an altered lipid metabolism, it seems that DAPP significantly improves mitochondrial dysfunction while displaying positive effects on NAFLD.

LPS is produced by gram-negative bacteria and is presently regarded as a central player in NAFLD pathogenesis and complication [71]. Its incorporation into the liver elicits release of inflammatory mediators, induces IR and causes organ damage [72,73]. The high concentrations in the liver and blood circulation were associated with various metabolic disturbances. However, DAPP administration decreased LPS and ameliorated HE diet-induced disturbances, including hepatosteatosis. One mechanism for this action of DAPP was the interference with LPS transport through the inhibition of CM assembly and secretion. Therefore, these findings reveal novel mechanisms of action of DAPP against LPS bioavailability, which translates into improvement of LPS-induced liver injury.

In line with several studies, the administration of HE diet to P. obesus was associated with loosening of intestinal tight junctions, increased intestinal permeability and elevated systemic levels of LPS [74,75]. Since a direct link was reported between these abnormalities, which contribute to MetS-related comorbidities such as NAFLD, and intestinal dysbiosis, we measured SCFA that are produced by gut microbial fermentation and serve both as an energy source and signaling molecules. For example, SCFAs have been shown to inhibit lipogenesis by acting on the transcription of several rate-limiting enzymes involved in de novo lipogenesis, namely ACC and FAS [76,77]. Interestingly, the treatment of insulin-resistant P. obesus with DAPP led to increased SCFA concentration in the gut, which can inhibit lipogenesis, limit OxS and inflammation, thereby protecting against liver damage via modifications of intestinal microbiota [5,78]. It is therefore possible that DAPP exert prebiotic effects in insulin-resistant P. obesus. The rise of SCFA, reduction of LPS and TMAO, lessening of permeability and reinforcement of tight junctions in response to DAPP treatment are evocative of microbiota involvement.

In conclusion, DAPP exhibit a protective role against NAFLD in P. obesus gerbils fed a HE diet. Their beneficial effects were mediated by critical mechanisms among which the improvement of intestinal barrier integrity, endotoxemia and SCFA metabolites should be highlighted. As these specific parameters generally result from resident microbiota modulation, it may be suggested that DAPP exert prebiotic effects protecting both the intestine and liver against CMD.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCES

1. European Association for the Study of the, L.; European Association for the Study of, D.; European Association for the Study of, O. EASL-EASD-EASO Clinical Practice Guidelines for the management of non-alcoholic fatty liver disease. J Hepatol 2016, 64, 1388-1402.
2. Non-alcoholic Fatty Liver Disease Study, G.; Lonardo, A.; Bellentani, S.; Argo, C. K.; Ballestri, S.; Byrne, C. D.; Caldwell, S. H.; Cortez-Pinto, H.; Grieco, A.; Machado, M. V., et al. Epidemiological modifiers of non-alcoholic fatty liver disease: Focus on high-risk groups. Dig Liver Dis 2015, 47, 997-1006.
3. Younossi, Z. M.; Koenig, A. B.; Abdelatif, D.; Fazel, Y.; Henry, L.; Wymer, M. Global epidemiology of nonalcoholic fatty liver disease-Meta-analytic assessment of prevalence, incidence, and outcomes. Hepatology 2016, 64, 73-84.
4. Kotronen, A.; Yki-Jarvinen, H. Fatty liver: a novel component of the metabolic syndrome. Arterioscler Thromb Vasc Biol 2008, 28, 27-38.
5. Tilg, H.; Moschen, A. R.; Roden, M. NAFLD and diabetes mellitus. Nat Rev Gastroenterol Hepatol 2017, 14, 32-42.
6. Angulo, P. Nonalcoholic fatty liver disease. N Engl J Med 2002, 346, 1221-1231.
7. Hallsworth, K.; Hollingsworth, K. G.; Thoma, C.; Jakovljevic, D.; MacGowan, G. A.; Anstee, Q. M.; Taylor, R.; Day, C. P.; Trenell, M. I. Cardiac structure and function are altered in adults with non-alcoholic fatty liver disease. J Hepatol 2013, 58, 757-762.
8. Kolkailah, A. A.; Hirji, S. A.; Ejiofor, J. I.; Ramirez Del Val, F.; Lee, J.; Norman, A. V.; McGurk, S.; Mahmood, S.; Shook, D.; Vlassakov, K., et al. Novel fast-track recovery protocol for alternative access transcatheter aortic valve replacement: application to non-femoral approaches. Interact Cardiovasc Thorac Surg 2018, 26, 938-943.
9. Targher, G.; Day, C. P.; Bonora, E. Risk of cardiovascular disease in patients with nonalcoholic fatty liver disease. N Engl J Med 2010, 363, 1341-1350.
10. Gaggini, M.; Morelli, M.; Buzzigoli, E.; DeFronzo, R. A.; Bugianesi, E.; Gastaldelli, A. Non-alcoholic fatty liver disease (NAFLD) and its connection with insulin resistance, dyslipidemia, atherosclerosis and coronary heart disease. Nutrients 2013, 5, 1544-1560.
11. Marcuccilli, M.; Chonchol, M. NAFLD and Chronic Kidney Disease. Int J Mol Sci 2016, 17, 562.
12. Spahis, S.; Delvin, E.; Borys, J. M.; Levy, E. Oxidative Stress as a Critical Factor in Nonalcoholic Fatty Liver Disease Pathogenesis. Antioxid Redox Signal 2017, 26, 519-541.
13. Archuleta, T. L.; Lemieux, A. M.; Saengsirisuwan, V.; Teachey, M. K.; Lindborg, K. A.; Kim, J. S.; Henriksen, E. J. Oxidant stress-induced loss of IRS-1 and IRS-2 proteins in rat skeletal muscle: role of p38 MAPK. Free Radic Biol Med 2009, 47, 1486-1493.
14. Hotamisligil, G. S.; Peraldi, P.; Budavari, A.; Ellis, R.; White, M. F.; Spiegelman, B. M. IRS-1-mediated inhibition of insulin receptor tyrosine kinase activity in TNF-alpha- and obesity-induced insulin resistance. Science 1996, 271, 665-668.
15. Chen, Z.; Yu, R.; Xiong, Y.; Du, F.; Zhu, S. A vicious circle between insulin resistance and inflammation in nonalcoholic fatty liver disease. Lipids Health Dis 2017, 16, 203.
16. Issa, D.; Wattacheril, J.; Sanyal, A. J. Treatment options for nonalcoholic steatohepatitis—a safety evaluation. Expert Opin Drug Saf 2017, 16, 903-913.
17. Hannah, W. N., Jr.; Harrison, S. A. Lifestyle and Dietary Interventions in the Management of Nonalcoholic Fatty Liver Disease. Dig Dis Sci 2016, 61, 1365-1374.
18. Vilar-Gomez, E.; Martinez-Perez, Y.; Calzadilla-Bertot, L.; Torres-Gonzalez, A.; Gra-Dramas, B.; Gonzalez-Fabian, L.; Friedman, S. L.; Diago, M.; Romero-Gomez, M. Weight Loss Through Lifestyle Modification Significantly Reduces Features of Nonalcoholic Steatohepatitis. Gastroenterology 2015, 149, 367-378 e365; quiz e314-365.
19. Andersen, T.; Gluud, C.; Franzmann, M. B.; Christoffersen, P. Hepatic effects of dietary weight loss in morbidly obese subjects. J Hepatol 1991, 12, 224-229.
20. Anhe, F. F.; Roy, D.; Pilon, G.; Dudonne, S.; Matamoros, S.; Varin, T. V.; Garofalo, C.; Moine, Q.; Desjardins, Y.; Levy, E., et al. A polyphenol-rich cranberry extract protects from diet-induced obesity, insulin resistance and intestinal inflammation in association with increased Akkermansia spp. population in the gut microbiota of mice. Gut 2015, 64, 872-883.
21. Denis, M. C.; Roy, D.; Yeganeh, P. R.; Desjardins, Y.; Varin, T.; Haddad, N.; Amre, D.; Sane, A. T.; Garofalo, C.; Furtos, A., et al. Apple peel polyphenols: a key player in the prevention and treatment of experimental inflammatory bowel disease. Clin Sci (Lond) 2016, 130, 2217-2237.
22. Anhe, F. F.; Varin, T. V.; Le Barz, M.; Pilon, G.; Dudonne, S.; Trottier, J.; St-Pierre, P.; Harris, C. S.; Lucas, M.; Lemire, M., et al. Arctic berry extracts target the gut-liver axis to alleviate metabolic endotoxaemia, insulin resistance and hepatic steatosis in diet-induced obese mice. Diabetologia 2018, 61, 919-931.
23. Paolella, G.; Mandato, C.; Pierri, L.; Poeta, M.; Di Stasi, M.; Vajro, P. Gut-liver axis and probiotics: their role in non-alcoholic fatty liver disease. World J Gastroenterol 2014, 20, 15518-15531.
24. Zoltowska, M.; Ziv, E.; Delvin, E.; Stan, S.; Bar-On, H.; Kalman, R.; Levy, E. Circulating lipoproteins and hepatic sterol metabolism in Psammomys obesus prone to obesity, hyperglycemia and hyperinsulinemia. Atherosclerosis 2001, 157, 85-96.
25. Harmel, E.; Grenier, E.; Bendjoudi Ouadda, A.; El Chebly, M.; Ziv, E.; Beaulieu, J. F.; Sane, A.; Spahis, S.; Laville, M.; Levy, E. AMPK in the small intestine in normal and pathophysiological conditions. Endocrinology 2014, 155, 873-888.
26. Levy, E.; Lalonde, G.; Delvin, E.; Elchebly, M.; Precourt, L. P.; Seidah, N. G.; Spahis, S.; Rabasa-Lhoret, R.; Ziv, E. Intestinal and hepatic cholesterol carriers in diabetic Psammomys obesus. Endocrinology 2010, 151, 958-970.
27. Ben Djoudi Ouadda, A.; Levy, E.; Ziv, E.; Lalonde, G.; Sane, A. T.; Delvin, E.; Elchebly, M. Increased hepatic lipogenesis in insulin resistance and Type 2 diabetes is associated with AMPK signalling pathway up-regulation in Psammomys obesus. Biosci Rep 2009, 29, 283-292.
28. Levy, E.; Spahis, S.; Ziv, E.; Marette, A.; Elchebly, M.; Lambert, M.; Delvin, E. Overproduction of intestinal lipoprotein containing apolipoprotein B-48 in Psammomys obesus: impact of dietary n-3 fatty acids. Diabetologia 2006, 49, 1937-1945.
29. Zoltowska, M.; Delvin, E.; Ziv, E.; Peretti, N.; Chartre, M.; Levy, E. Impact of in vivo glycation of LDL on platelet aggregation and monocyte chemotaxis in diabetic psammomys obesus. Lipids 2004, 39, 81-85.
30. Zoltowska, M.; Ziv, E.; Delvin, E.; Lambert, M.; Seidman, E.; Levy, E. Both insulin resistance and diabetes in Psammomys obesus upregulate the hepatic machinery involved in intracellular VLDL assembly. Arterioscler Thromb Vasc Biol 2004, 24, 118-123.
31. Zoltowska, M.; Ziv, E.; Delvin, E.; Sinnett, D.; Kalman, R.; Garofalo, C.; Seidman, E.; Levy, E. Cellular aspects of intestinal lipoprotein assembly in Psammomys obesus: a model of insulin resistance and type 2 diabetes. Diabetes 2003, 52, 2539-2545.
32. Zoltowska, M.; St-Louis, J.; Ziv, E.; Sicotte, B.; Delvin, E. E.; Levy, E. Vascular responses to alpha-adrenergic stimulation and depolarization are enhanced in insulin-resistant and diabetic Psammomys obesus. Can J Physiol Pharmacol 2003, 81, 704-710.
33. Bendayan, M.; Malide, D.; Ziv, E.; Levy, E.; Ben-Sasson, R.; Kalman, R.; Bar-On, H.; Chretien, M.; Seidah, N. Immunocytochemical investigation of insulin secretion by pancreatic beta-cells in control and diabetic Psammomys obesus. J Histochem Cytochem 1995, 43, 771-784.
34. Kalderon, B.; Gutman, A.; Levy, E.; Shafrir, E.; Adler, J. H. Characterization of stages in development of obesity-diabetes syndrome in sand rat (Psammomys obesus). Diabetes 1986, 35, 717-724.
35. Adler, J. H.; Lazarovici, G.; Marton, M.; Levy, E. The diabetic response of weanling sand rats (Psammomys obesus) to diets containing different concentrations of salt bush (Atriplex halimus). Diabetes Res 1986, 3, 169-171.
36. Kalderon, B.; Adler, J. H.; Levy, E.; Gutman, A. Lipogenesis in the sand rat (Psammomys obesus). Am J Physiol 1983, 244, E480-486.
37. Yeganeh, P. R.; Leahy, J.; Spahis, S.; Patey, N.; Desjardins, Y.; Roy, D.; Delvin, E.; Garofalo, C.; Leduc-Gaudet, J. P.; St-Pierre, D., et al. Apple peel polyphenols reduce mitochondrial dysfunction in mice with DSS-induced ulcerative colitis. J Nutr Biochem 2018, 57, 56-66.
38. Denis, M. C.; Furtos, A.; Dudonne, S.; Montoudis, A.; Garofalo, C.; Desjardins, Y.; Delvin, E.; Levy, E. Apple peel polyphenols and their beneficial actions on oxidative stress and inflammation. PLoS One 2013, 8, e53725.
39. Maislos, M.; Medvedovskv, V.; Sztarkier, I.; Yaari, A.; Sikuler, E. Psammomys obesus (sand rat), a new animal model of non-alcoholic fatty liver disease. Diabetes Res Clin Pract 2006, 72, 1-5.
40. Spolding, B.; Connor, T.; Wittmer, C.; Abreu, L. L.; Kaspi, A.; Ziemann, M.; Kaur, G.; Cooper, A.; Morrison, S.; Lee, S., et al. Rapid development of non-alcoholic steatohepatitis in Psammomys obesus (Israeli sand rat). PLoS One 2014, 9, e92656.
41. Levy, E.; Thibault, L.; Menard, D. Intestinal lipids and lipoproteins in the human fetus: modulation by epidermal growth factor. J Lipid Res 1992, 33, 1607-1617.
42. Levy, E.; Menard, D.; Delvin, E.; Stan, S.; Mitchell, G.; Lambert, M.; Ziv, E.; Feoli-Fonseca, J. C.; Seidman, E. The polymorphism at codon 54 of the FABP2 gene increases fat absorption in human intestinal explants. J Biol Chem 2001, 276, 39679-39684.
43. Ferretti, E.; Tremblay, E.; Thibault, M. P.; Grynspan, D.; Burghardt, K. M.; Bettolli, M.; Babakissa, C.; Levy, E.; Beaulieu, J. F. The nitric oxide synthase 2 pathway is targeted by both pro- and anti-inflammatory treatments in the immature human intestine. Nitric Oxide 2017, 66, 53-61.
44. Perron, N.; Tremblay, E.; Ferretti, E.; Babakissa, C.; Seidman, E. G.; Levy, E.; Menard, D.; Beaulieu, J. F. Deleterious effects of indomethacin in the mid-gestation human intestine. Genomics 2013, 101, 171-177.
45. Menard, D.; Tremblay, E.; Ferretti, E.; Babakissa, C.; Perron, N.; Seidman, E. G.; Levy, E.; Beaulieu, J. F. Anti-inflammatory effects of epidermal growth factor on the immature human intestine. Physiol Genomics 2012, 44, 268-280.
46. Levy, E.; Delvin, E.; Menard, D.; Beaulieu, J. F. Functional development of human fetal gastrointestinal tract. Methods Mol Biol 2009, 550, 205-224.
47. Levy, E.; Menard, D.; Suc, I.; Delvin, E.; Marcil, V.; Brissette, L.; Thibault, L.; Bendayan, M. Ontogeny, immunolocalisation, distribution and function of SR-BI in the human intestine. J Cell Sci 2004, 117, 327-337.
48. Levy, E.; Stan, S.; Garofalo, C.; Delvin, E. E.; Seidman, E. G.; Menard, D. Immunolocalization, ontogeny, and regulation of microsomal triglyceride transfer protein in human fetal intestine. Am J Physiol Gastrointest Liver Physiol 2001, 280, G563-571.
49. Loirdighi, N.; Menard, D.; Delvin, D.; Levy, E. Selective effects of hydrocortisone on intestinal lipoprotein and apolipoprotein synthesis in the human fetus. J Cell Biochem 1997, 66, 65-76.
50. Slight, I.; Bendayan, M.; Malo, C.; Delvin, E.; Lambert, M.; Levy, E. Identification of microsomal triglyceride transfer protein in intestinal brush-border membrane. Exp Cell Res 2004, 300, 11-22.
51. Mohan, S.; Patel, H.; Bolinaga, J.; Soekamto, N. AMP-activated protein kinase regulates L-arginine mediated cellular responses. Nutr Metab (Lond) 2013, 10, 40.
52. Volynets, V.; Reichold, A.; Bardos, G.; Rings, A.; Bleich, A.; Bischoff, S. C. Assessment of the Intestinal Barrier with Five Different Permeability Tests in Healthy C57BL/6J and BALB/cJ Mice. Dig Dis Sci 2016, 61, 737-746.
53. Purandare, S.; Offenbartl, K.; Westrom, B.; Bengmark, S. Increased gut permeability to fluorescein isothiocyanate-dextran after total parenteral nutrition in the rat. Scand J Gastroenterol 1989, 24, 678-682.
54. Levy, E.; Trudel, K.; Bendayan, M.; Seidman, E.; Delvin, E.; Elchebly, M.; Lavoie, J. C.; Precourt, L. P.; Amre, D.; Sinnett, D. Biological role, protein expression, subcellular localization, and oxidative stress response of paraoxonase 2 in the intestine of humans and rats. Am J Physiol Gastrointest Liver Physiol 2007, 293, G1252-1261.
55. Marcil, V.; Lavoie, J. C.; Emonnot, L.; Seidman, E.; Levy, E. Analysis of the effects of iron and vitamin C co-supplementation on oxidative damage, antioxidant response and inflammation in THP-1 macrophages. Clin Biochem 2011, 44, 873-883.
56. Taha, R.; Seidman, E.; Mailhot, G.; Boudreau, F.; Gendron, F. P.; Beaulieu, J. F.; Menard, D.; Delvin, E.; Amre, D.; Levy, E. Oxidative stress and mitochondrial functions in the intestinal Caco-2/15 cell line. PLoS One 2010, 5, e11817.
57. Kleme, M. L.; Sane, A.; Garofalo, C.; Seidman, E.; Brochiero, E.; Berthiaume, Y.; Levy, E. CFTR Deletion Confers Mitochondrial Dysfunction and Disrupts Lipid Homeostasis in Intestinal Epithelial Cells. Nutrients 2018, 10.
58. Lambert, J. E.; Ramos-Roman, M. A.; Browning, J. D.; Parks, E. J. Increased de novo lipogenesis is a distinct characteristic of individuals with nonalcoholic fatty liver disease. Gastroenterology 2014, 146, 726-735.
59. Ghoshal, S.; Witta, J.; Zhong, J.; de Villiers, W.; Eckhardt, E. Chylomicrons promote intestinal absorption of lipopolysaccharides. J Lipid Res 2009, 50, 90-97.
60. Kitabatake, H.; Tanaka, N.; Fujimori, N.; Komatsu, M.; Okubo, A.; Kakegawa, K.; Kimura, T.; Sugiura, A.; Yamazaki, T.; Shibata, S., et al. Association between endotoxemia and histological features of nonalcoholic fatty liver disease. World J Gastroenterol 2017, 23, 712-722.
61. Li, Y.; Lu, Z.; Ru, J. H.; Lopes-Virella, M. F.; Lyons, T. J.; Huang, Y. Saturated Fatty Acid Combined with Lipopolysaccharide Stimulates a Strong Inflammatory Response in Hepatocytes in vivo and in vitro. Am J Physiol Endocrinol Metab 2018.
62. Zigmond, E.; Tayer-Shifman, O.; Lalazar, G.; Ben Ya'acov, A.; Weksler-Zangen, S.; Shasha, D.; Sklair-Levy, M.; Zolotarov, L.; Shalev, Z.; Kalman, R., et al. beta-glycosphingolipids ameliorated non-alcoholic steatohepatitis in the Psammomys obesus model. J Inflamm Res 2014, 7, 151-158.
63. Neuschwander-Tetri, B. A. Nonalcoholic steatohepatitis and the metabolic syndrome. Am J Med Sci 2005, 330, 326-335.
64. Younossi, Z.; Henry, L. Contribution of Alcoholic and Nonalcoholic Fatty Liver Disease to the Burden of Liver-Related Morbidity and Mortality. Gastroenterology 2016, 150, 1778-1785.
65. Samuel, V. T.; Liu, Z. X.; Qu, X.; Elder, B. D.; Bilz, S.; Befroy, D.; Romanelli, A. J.; Shulman, G. I. Mechanism of hepatic insulin resistance in non-alcoholic fatty liver disease. J Biol Chem 2004, 279, 32345-32353.
66. Sozio, M. S.; Lu, C.; Zeng, Y.; Liangpunsakul, S.; Crabb, D. W. Activated AMPK inhibits PPAR-{alpha} and PPAR-{gamma} transcriptional activity in hepatoma cells. Am J Physiol Gastrointest Liver Physiol 2011, 301, G739-747.
67. Srivastava, R. A.; Pinkosky, S. L.; Filippov, S.; Hanselman, J. C.; Cramer, C. T.; Newton, R. S. AMP-activated protein kinase: an emerging drug target to regulate imbalances in lipid and carbohydrate metabolism to treat cardio-metabolic diseases. J Lipid Res 2012, 53, 2490-2514.
68. Mihaylova, M. M.; Shaw, R. J. The AMPK signalling pathway coordinates cell growth, autophagy and metabolism. Nat Cell Biol 2011, 13, 1016-1023.
69. Rolo, A. P.; Teodoro, J. S.; Palmeira, C. M. Role of oxidative stress in the pathogenesis of nonalcoholic steatohepatitis. Free Radic Biol Med 2012, 52, 59-69.
70. Begriche, K.; Igoudjil, A.; Pessayre, D.; Fromenty, B. Mitochondrial dysfunction in NASH: causes, consequences and possible means to prevent it. Mitochondrion 2006, 6, 1-28.
71. Ceccarelli, S.; Panera, N.; Mina, M.; Gnani, D.; De Stefanis, C.; Crudele, A.; Rychlicki, C.; Petrini, S.; Bruscalupi, G.; Agostinelli, L., et al. LPS-induced TNF-alpha factor mediates pro-inflammatory and pro-fibrogenic pattern in non-alcoholic fatty liver disease. Oncotarget 2015, 6, 41434-41452.
72. Frasinariu, O. E.; Ceccarelli, S.; Alisi, A.; Moraru, E.; Nobili, V. Gut-liver axis and fibrosis in nonalcoholic fatty liver disease: an input for novel therapies. Dig Liver Dis 2013, 45, 543-551.
73. Miura, K.; Seki, E.; Ohnishi, H.; Brenner, D. A. Role of toll-like receptors and their downstream molecules in the development of nonalcoholic Fatty liver disease. Gastroenterol Res Pract 2010, 2010, 362847.
74. Cani, P. D.; Possemiers, S.; Van de Wiele, T.; Guiot, Y.; Everard, A.; Rottier, O.; Geurts, L.; Naslain, D.; Neyrinck, A.; Lambert, D. M., et al. Changes in gut microbiota control inflammation in obese mice through a mechanism involving GLP-2-driven improvement of gut permeability. Gut 2009, 58, 1091-1103.
75. Cani, P. D.; Bibiloni, R.; Knauf, C.; Waget, A.; Neyrinck, A. M.; Delzenne, N. M.; Burcelin, R. Changes in gut microbiota control metabolic endotoxemia-induced inflammation in high-fat diet-induced obesity and diabetes in mice. Diabetes 2008, 57, 1470-1481.
76. Lin, Y.; Vonk, R. J.; Slooff, M. J.; Kuipers, F.; Smit, M. J. Differences in propionate-induced inhibition of cholesterol and triacylglycerol synthesis between human and rat hepatocytes in primary culture. Br J Nutr 1995, 74, 197-207.
77. Tilg, H.; Cani, P. D.; Mayer, E. A. Gut microbiome and liver diseases. Gut 2016, 65, 2035-2044.
78. Postler, T. S.; Ghosh, S. Understanding the Holobiont: How Microbial Metabolites Affect Human Health and Shape the Immune System. Cell Metab 2017, 26, 110-130.

1. An apple peel polyphenolic extract comprising
a proanthocyanidin (PAC) content of at least 15000 μg/g of dry weight, comprising
an about 30% to about 35% epicatechin content comprising
epicatechin momomers and oligomers having from about 1 to 5 epicatechin units, from about 3500 to 4000 μg/g of dry weight;
epicatechin oligomers having from about 6 to 10 epicatechin units, from about 1000 to 1100 μg/g of dry weight; and
epicatechin polymers having >10 epicatechin units, from about 500 to 600 μg/g of dry weight.

2. The apple peel polyphenolic extract of claim 1, wherein said epicatechin momomers and oligomers having from about 1 to 5 epicatechin units comprises epicatechin monomers, from about 700 to 800 μg/g of dry weight.

3. The apple peel polyphenolic extract of claim 1, wherein said epicatechin momomers and oligomers having from about 1 to 5 epicatechin units comprises epicatechin dimers, from about 1000 to about 1200 μg/g of dry weight.

4. The apple peel polyphenolic extract of claim 1, wherein said epicatechin momomers and oligomers having from about 1 to 5 epicatechin units comprises epicatechin trimers, from about 600 to 700 μg/g of dry weight.

5. The apple peel polyphenolic extract of claim 1, wherein said epicatechin momomers and oligomers having from about 1 to 5 epicatechin units comprises epicatechin tetramers, from about 500 to 600 μg/g of dry weight.

6. The apple peel polyphenolic extract of claim 1, wherein said epicatechin momomers and oligomers having from about 1 to 5 epicatechin units comprises epicatechin pentamers, from about 500 to 600 μg/g of dry weight.

7. The apple peel polyphenolic extract of claim 1, further comprising a total flavonol content of from about 5000 to about 5400 μg/g of dry weight.

8. The apple peel polyphenolic extract of claim 7, wherein said total flavonol content comprises at least one of:
1) a quercetin-glucoside, from about 1600 to about 2000 μg/g of dry weight;
2) a quercetin-arabinoside, from about 950 to about 1150 μg/g of dry weight;
3) a quercetin-xyloside, from about 500 to about 600 μg/g of dry weight;
4) a quercetin-galactoside, from about 300 to about 400 μg/g of dry weight;
5) a quercetin-rhamnoside, from about 1100 to about 1300 μg/g of dry weight;
6) quercetin, from about 100 to about 200 μg/g of dry weight.

9.-13. (canceled)

14. The apple peel polyphenolic extract of claim 7, wherein said total flavonol content comprises a rutin, from about 200 to about 300 μg/g of dry weight.

15. The apple peel polyphenolic extract of claim 7, wherein said total flavonol content comprises a phlorizin, from about 1200 to about 1400 μg/g of dry weight.

16. The apple peel polyphenolic extract of claim 1, further comprising a total phenolic acid content of from about 2000 to about 2400 μg/g of dry weight.

17. The apple peel polyphenolic extract of claim 16, wherein said total phenolic acid content comprises at least one of
1) a chlorogenic acid, from about 2000 to about 2100 μg/g of dry weight;
2) a coumaroyl glucoside, from about 10 to about 20 μg/g of dry weight;
3) a dihydrobenzoic acid, from about 25 to about 45 μg/g of dry weight;
4) a caffeic acid, from about 1 to about 10 μg/g of dry weight; and
5) a coumaroyl hexose, from about 100 to about 200 μg/g of dry weight.

18.-21. (canceled)

22. The apple peel polyphenolic extract of claim 1, comprising:
a proanthocyanidin (PAC) content of at least 15000 μg/g of dry weight, comprising
an about 30% to about 35% epicatechin content comprising
epicatechin oligomers having from about 1 to 5 epicatechin units, from about 3500 to 4000 μg/g of dry weight;
epicatechin oligomers having from about 6 to 10 epicatechin units, from about 1000 to 1100 μg/g of dry weight; and
epicatechin polymers having >10 epicatechin units, from about 500 to 600 μg/g of dry weight;
a total flavonol content of from about 5000 to about 5400 μg/g of dry weight; and
a total phenolic acid content of from about 2000 to about 2400 μg/g of dry weight.

23. A pharmaceutical composition comprising the apple peel polyphenolic extract of claim 1, and a pharmaceutically acceptable carrier.

24. A method of preventing or treating oxidative stress, inflammation and mitochondrial dysfunction of a liver comprising administering to a subject in need thereof a therapeutically effective amount of an apple peel polyphenolic extract according to any one of claim 1.

25. A method of inhibition of de novo lipogenesis and β-oxidation of free fatty acids for preventing accumulation thereof in a liver comprising administering to a subject in need thereof a therapeutically effective amount of an apple peel polyphenolic extract according to claim 1.

26. A method of preventing or treating insulin resistance comprising administering to a subject in need thereof a therapeutically effective amount of an apple peel polyphenolic extract according to claim 1.

27. A method of preventing or treating intestinal endothelial tissue injury comprising administering to a subject in need thereof a therapeutically effective amount of an apple peel polyphenolic extract according to claim 1.

28. (canceled)

29. A method of preventing or treating any one of non-alcoholic fatty liver disease, non-alcoholic steatohepatitis (NASH), liver fibrosis, liver cirrhosis, and combinations thereof comprising administering to a subject in need thereof a therapeutically effective amount of an apple peel polyphenolic extract according to claim 1.

30. The method of claim 29, wherein said non-alcoholic fatty liver disease, non-alcoholic steatohepatitis (NASH), liver fibrosis, and liver cirrhosis comprises steatosis.

31. The method of claim 29, wherein said therapeutically effective amount is from about 100 mg/kg to about 500 mg/kg.

32. The method of claim 29, wherein said apple peel polyphenolic extract is for once daily use, twice daily use, thrice daily use.

33. The method of claim 29, wherein said subject is a human subject.

34.-70. (canceled)

71. A method of preventing or treating any one of non-alcoholic fatty liver disease, non-alcoholic steatohepatitis (NASH), liver fibrosis, liver cirrhosis, and combinations thereof comprising administering to a subject in need thereof a therapeutically effective amount of a pharmaceutical composition according to claim 23.

72. A method of preventing or treating oxidative stress, inflammation and mitochondrial dysfunction of a liver comprising administering to a subject in need thereof a therapeutically effective amount of a pharmaceutical composition according to claim 23.

73. A method of inhibition of de novo lipogenesis and β-oxidation of free fatty acids for preventing accumulation thereof in a liver comprising administering to a subject in need thereof a therapeutically effective amount of a pharmaceutical composition according to claim 23.

74. A method of preventing or treating insulin resistance comprising administering to a subject in need thereof a therapeutically effective amount of a pharmaceutical composition according to claim 23.

75. A method of preventing or treating intestinal endothelial tissue injury comprising administering to a subject in need thereof a therapeutically effective amount of a pharmaceutical composition according to claim 23.

76. A method of preventing or treating any one of non-alcoholic fatty liver disease, non-alcoholic steatohepatitis (NASH), liver fibrosis, liver cirrhosis, and combinations thereof comprising administering to a subject in need thereof a therapeutically effective amount of a pharmaceutical composition according to claim 23.


SEQUENCE LISTING (1 sequence)

SEQ ID NO: 1 - protein, 15 aa, Artificial Sequence
  synthetic SAMS peptide substrate
HMRSAMSGLHLVKRR